(19) United States
(12) Patent Application Publication
(10) Pub. No.: US 2004/0170614 A1
(43) Pub. Date: Sep. 2, 2004

(54) Somatic cell gene targeting vectors and methods of use thereof

(76) Inventors: Gail Bishop, Iowa City, IA (US); Bruce S. Hostager, Coralville, IA (US)

Correspondence Address: FISH & RICHARDSON P.C., 3300 DAIN RAUSCHER PLAZA, 60 SOUTH SIXTH STREET, MINNEAPOLIS, MN, 55402, US

(21) Appl. No.: 10/694,520

(22) Filed: Oct. 27, 2003

Related U.S. Application Data
(60) Provisional application No. 60/422,674, filed on Oct. 30, 2002.

(51) Int. Cl. A61K 48/00; C12N 5/08

(52) U.S. Cl. 424/93.21; 435/320.1; 435/366

(57) ABSTRACT

The present invention provides vectors for gene targeting and disruption in somatic cells, and methods for using the same.

### CLAIM OF PRIORITY

This application claims priority under 35 U.S.C. 119(e) from U.S. Provisional Patent Application Serial No. 60/422,674 filed Oct. 30, 2002, the entirety of which is incorporated by reference.

### STATEMENT OF GOVERNMENT RIGHTS

The invention was made with the support of NIH Grant numbers RO1 AI 28847 and RO1 AI 213300. the U.S. Government has certain rights in the invention.

### BACKGROUND OF THE INVENTION

Gene targeting technology allows the exchange of an endogenous allele of a target gene for an exogenous copy via homologous recombination, and is a common approach for studying the effects of deleting a gene of interest on cell function. However, current methods for targeting genes for disruption by homologous recombination require the use of cells that inherently have a high rate of homologous recombination, e.g., germ cells.

The most popular approach for gene disruption in somatic cell lines has been to treat a whole culture of cells with a mutagenic drug, then attempt to screen large numbers to find a subclone in which the gene product no longer functions normally. This approach has substantial drawbacks, including the fact that the mutagen may alter unknown functions in the cell. Moreover, in many cases gene function is decreased using this method, but gene expression is not eliminated. Additionally, screening can only identify a defective process. Once the process is identified, the investigator must then do a considerable amount of work to find the gene involved, which sometimes is never found.

While potentially a valuable tool for evaluating the roles of a variety of cellular proteins, targeted disruption of genes in somatic cell lines has been used infrequently. This is due, in part, to the fact that the absolute frequency of homologous recombination in somatic cells is approximately two orders of magnitude lower than in embryonic stem cells (Brown et al., 1997; Arbones et al., 1994; Hanson and Sedivy, 1995).

### SUMMARY OF THE INVENTION

Using the vectors and methods described herein, the expression of a particular gene in a somatic cell can be disrupted by a targeted, i.e., directed recombination event, for example, by targeted homologous recombination. The methods described herein require much less time and expense than known methods for gene disruption in somatic cells. In addition, the vectors and methods described herein allow for the rapid transfection of cells with desired molecules, thus facilitating the ability to test hypotheses and predictions. Moreover, the vectors and methods described herein target genes specifically and avoid the production of additional unknown and undesired mutations, a common result when using chemical mutagens to disrupt somatic cell genes.

The present invention provides a somatic cell gene targeting vector that has a gene targeting construct containing a first cloning site operably linked to a DNA encoding a positive selection marker, for example, neomycin phosphotransferase, a second cloning site and a first polyadenylation sequence, for example, a SV40 polyadenylation sequence. The construct is promoterless. The vector also contains an expression cassette having a promoter operably linked to DNA encoding a negative selection marker and a second polyadenylation sequence, e.g., a BGH polyadenylation sequence. The gene targeting construct can further contain site-specific recombination sequences for a recombinase. For example, the recombinase can be Cre recombinase, and the site-specific recombination sequences can be loxP sequences. The site-specific recombination sequences flank the DNA encoding the positive selection marker. In addition, the gene targeting construct may have a first cloning site containing a first DNA segment that is homologous to a first genomic target sequence (i.e., a 5′-genomic flank), and a second cloning site containing a second DNA segment that is homologous to a second genomic target sequence (i.e., a 3′-genomic flank). The promoter of the expression cassette can be a weak promoter. The promoter can be a phosphoglycerate kinase (PGK) promoter or a modified Rous sarcoma virus (RSV) promoter. In one embodiment of the invention, the promoter is a modified RSV promoter. The negative selection marker of the expression cassette can be HSV thymidine kinase or diphtheria toxin (DT-A).

Further provided is a method for disrupting a gene of interest in a somatic cell, which involves introducing such a vector into a somatic cell, e.g., a somatic mammalian cell such as a human cell, such that a portion of the vector recombines with the gene to yield a genetically altered cell. Sequences in the vector can recombine with the gene, for example, via a homologous recombination event. Further, the method can involve identifying the genetically altered cell, wherein the cell's genome includes the construct and the positive selection marker is expressed. In addition, a double-stranded oligonucleotide, e.g., a 62 bp double-stranded oligonucleotide, can be introduced into the cell.

Further provided is a method for disrupting a gene of interest in a somatic cell, which involves introducing a vector having a promoterless gene targeting construct that has a first cloning site operably linked to a DNA encoding a positive selection marker, a second cloning site, a first polyadenylation sequence, a first site-specific recombination sequence for a recombinase and a second site-specific recombination sequence for the recombinase, wherein the first and second site-specific recombination sequences flank the DNA encoding the positive selection marker, as well as an expression cassette comprising a promoter operably linked to DNA encoding a negative selection marker and a second polyadenylation sequence. The vector is introduced into the somatic cell such that a portion of the vector recombines with the gene to yield a first genetically altered cell. Sequences in the vector can recombine with the gene by homologous recombination. The method further involves introducing a recombinase to the genetically altered cell, such that the positive selection marker is removed from the construct to yield a second genetically altered cell. The method can further involve identifying the first genetically altered cell, wherein the cell's genome includes the sequences from the construct and the positive selection marker is expressed. The method can additionally involve identifying the second genetically altered cell. Further, the method can involve introducing a double-stranded oligonucleotide, for example, a 62 bp double-stranded oligonucleotide, into the somatic cell. Further provided is an isolated cell prepared by such methods, and a somatic cell, such as a B cell or fibroblast cell, that contains a vector of the invention.

Abbreviations used herein are the following: CHX, cycloheximide; CY, cytoplasmic; DN, dominant-negative; hCD40, human CD40; hmCD40, human-mouse hybrid CD40; IPTG, isopropyl-β-D-thiogalactopyrano side; JNK, c-Jun kinase; mCD40, mouse CD40; Pfc, plaque-forming cell; pA, polyadenylation; SRBC, sheep red blood cell; TNFR, tumor necrosis factor receptor; TRAF, TNF receptor associated factor.

### BRIEF DESCRIPTION OF THE FIGURES

FIG. 1. Design and use of TRAF2 targeting constructs. (A) pPNTV1 (not shown) and pPNTV2 were constructed for the targeted disruption of genomic sequences. The two vectors differ only in the promoter used to drive diphtheria toxin (DTA) expression. Genomic sequences were inserted into the endonuclease restriction sites immediately upstream of loxP-NeoR, and into the SacI or KpnI sites downstream of the SV40 pA site. (B) Genomic DNA sequences used in each of three targeting vectors are shown with a map of the TRAF2 gene for reference. Genomic segments positioned upstream of NeoR in the targeting vectors are shown as black bars; the white bars show the segments used for downstream (3′) flanks. (C) Western blot analysis of whole-cell lysates demonstrates TRAF2-deficiency in targeted CH12.LX and A20.2J cell lines. Western blots were reprobed for actin expression to show similar lane loading. (D) Western blot analysis of whole-cell lysates illustrating TRAF2 expression in TRAF2-deficient CH12.LX cells reconstituted with an IPTG-inducible TRAF2 expression plasmid (CH 12.rT2). TRAF2 expression was induced with a 24 hr. incubation of the cells with 100 μM IPTG. Western blots were reprobed for TRAF3 expression to show similar lane loading. Similar results were obtained in 2 additional experiments.

FIG. 2. Defective CD40-stimulated TRAF3 degradation in TRAF2-deficient cells. (A) Total cell lysates were prepared from cells stimulated for six hours with an anti-mCD40 antibody (+) or isotype control Ab (−). Where indicated, CH12.LX cells were incubated in 1.0 μM cycloheximide (CHX) for 30 minutes prior to the experiment to inhibit new protein synthesis (this concentration of cycloheximide was found to inhibit TNF production by >90%, data not shown). Levels of TRAF3 and actin in each lysate were determined by Western blot. A separate Western blot for TRAF2 (from the same samples) appears below the actin blot. (B) TRAF3 degradation in TRAF2-deficient cells reconstituted with IPTG-inducible expression vectors encoding wild-type TRAF2 (CH12.rT2 cells) or DNTRAF2 (CH12.rDNT2 cells). Where indicated, cells were treated with 100 μM IPTG for 24 hrs prior to stimulation to induce TRAF2 expression. Cells were stimulated as in (A). (C) Quantitation of TRAF3 degradation. TRAF3 and actin bands on Western blots in (A) and (B) were quantitated using a low-light imaging system, and the results presented graphically. The amount of TRAF3 in each lane was normalized to the intensity of the corresponding actin band. The graph depicts the mean TRAF3 degradation observed in three experiments (±SEM). (D) Quantitation of TRAF3 degradation in A20.2J and TRAF2-deficient A20.2J cells (experiments performed as above; the graph depicts the mean TRAF3 degradation observed in three experiments±SEM).

FIG. 3. CD40-stimulated IgM secretion by TRAF2^{-/-} B cells. (A) IgM secretion by CH12.LX and CH12.T2^{-/-} cells stimulated with mCD154-expressing insect cells (Sf9-mCD154), control insect cells (Sf9) or 50 pg/ml TNF. The vertical axis indicates the number of plaque forming (antibody secreting) cells (Pfc) per 10⁶ viable recovered cells. Similar results were obtained in 4 additional experiments using mCD154, and 8 experiments using anti-mCD40 (1C10) as a CD40 stimulus. (B) Anti-mCD40 and TNF-stimulated IgM secretion by CH 12.T2^{-/-} cells reconstituted with IPTG-inducible TRAF2. Similar results were obtained in 4 additional experiments.

FIG. 4. Activation of IgM secretion by hmCD40ΔT6 in CH12.LX and CH12.T2^{-/-}. (A) Binding of TRAF6 to hmCD40 and hmCD40ΔT6 in B cells. CH12.LX cells stably expressing hmCD40 or hmCD40ΔT6 were stimulated for 20 min. with control insect cells (−) or insect cells expressing hCD 154 (+) to induce the association of CD40 with membrane microdomains and the association of TRAFs with CD40. Following stimulation, semi-purified microdomains were isolated, from which human CD40 was immunoprecipitated. Anti-hCD40 immunoprecipitates were examined by Western blotting for CD40-associated TRAF6. The membrane was stripped and reprobed for hCD40 to show equivalent immunoprecipitation and lane loading. Similar results were obtained in a second experiment. (B) CH12.LX and CH12.T2^{-/-} cells stably transfected with hmCD40 or hmCD40ΔT6 (ΔT6) were stimulated with anti-mCD40 to engage endogenous CD40 or anti-hCD40 to engage the transfected molecules. The isotype control was a mixture of the isotype control mAbs for anti-mCD40 and anti-hCD40. The response of cells to anti-hCD40 (stimulation through the transfected molecule) relative to the anti-mCD40 (endogenous CD40) response is presented. Error bars represent the range of duplicate samples. Actual Pfc values (±range of duplicate samples) for each condition were as follows: CH12.LX+hmCD40, isotype control: 18166.5±833.5, anti-mCD40: 431136±6136, anti-hCD40: 430469±37136; CH 12.LX+hmCD40ΔT6, isotype control: 3055±1389, anti-mCD40: 378450.5±28821.5, anti-hCD40:187412±8951; CH12.T2^{-/-}+hmCD40, isotype control: 2123±457, anti-mCD40: 398916.5±6416.5, anti-hCD40: 375555±11111; CH12.T2^{-/-}+hmCD40ΔT6, isotype control: 4272.5±272.5, anti-mCD40: 340000±28000, anti-hCD40: 34047.5±4047.5. Similar results were obtained in a second experiment.

FIG. 5. Defective BCR-CD40 synergy in TRAF2^{-/-} cells. (A) IgM secretion by CH12.LX and CH12.T2^{-/-} cells stimulated with Ag (SRBC), anti-mCD40, or both. (B) CD40/Ag-mediated IgM secretion by CH12.T2^{-/-} and CH12.T2^{-/-} cells reconstituted with IPTG-inducible FLAG-tagged TRAF2 (CH12.rT2F). (C) CD40/Ag-mediated IgM secretion by CH12.T2^{-/-} cells reconstituted with IPTG-inducible FLAG-tagged DNTRAF2. (D) CD40/Ag-mediated IgM secretion by CH12.T2^{-/-} cells transfected with hCD40Δ22. Similar results were obtained in four (A, B), three (C) or two (D) additional experiments.

FIG. 6. Activation of JNK in TRAF2^{-/-} cells. (A) Cells were stimulated for various lengths of time with anti-mCD40 (α-mCD40) or an isotype control Ab (I.C., 5 min. time point). Activation of JNK was determined by Western blot for the two phosphorylated isoforms (p54 and p46) of JNK (upper panel). Western blots were reprobed for total JNK to demonstrate similar lane loading (lower panel). (B) Activation of JNK in CH 12.T2^{-/-} cells transfected with inducible TRAF2. Where indicated, cells were incubated overnight with 100 μM IPTG prior to the experiment. Cells were stimulated and assayed as in (A). Similar results were obtained in a second experiment, and in four additional experiments using an in vitro kinase assay to measure JNK activity (not shown).

FIG. 7. NF-κB activation in TRAF2^{-/-} A20.2J cells. (A) Whole-cell lysates were prepared from unstimulated cells or cells stimulated for various times with anti-mCD40 mAb, or for 5 min. with an isotype control Ab (I.C.). Activation-induced phosphorylation of IκBa was detected by Western blotting. Membranes were stripped and reprobed with an Ab against total IκBα to show activation-induced degradation, and an anti-actin Ab to demonstrate equal lane loading. Similar results were obtained in a second experiment. (B) hmCD40ΔT6 remains able to induce NF-κB in cells expressing TRAF2. Cells were stimulated with anti-mCD40 or anti-hCD40 mAbs and assayed as in (A). Similar results were obtained in two additional experiments. (C) hmCD40ΔT6 has reduced ability to activate NF-κB in TRAF2^{-/-} cells. In contrast, cells remain responsive to stimulation through endogenous mCD40. Similar results were obtained in two additional experiments.

FIG. 8. CD80 upregulation in A20.2J and A20.T2^{-/-} cells by CD40 and hmCD40ΔT6. Cells were stimulated for 3 days with anti-mCD40 (to activate through endogenous mCD40) (8A, 8C, 8E, and 8G) or anti-hCD40 (to stimulate through hmCD40ΔT6) (8B, 8D, 8F, and 8H). CD80 expression of stimulated cells is presented in the filled profiles on the flow cytometry histograms. Open profiles indicate CD80 expression of cells stimulated with isotype control Abs. Similar results were obtained in a second experiment.

FIG. 9 depicts the position of TRAF binding sites in the cytoplasmic domain of human CD40.

FIG. 10 depicts PCR screening for homologous recombination of TRAF2 targeting vector in CH12.LX and NIH3T3 cells. (A) Diagram of TRAF2 targeting vector. Approximate positions of primers (rNeo, T2delA-5′S) used for screening are indicated by arrows. (B) G418-resistant clones of CH12.LX transfected with the pT2delC targeting vector were screened by PCR. Lane 1 is the PCR product generated from a clone containing homologous integration of the targeting vector. Disruption of TRAF2 expression in was verified by Western blot for TRAF2 protein after disruption of second allele of TRAF2. Lane 2 shows the PCR screening performed on a clone containing randomly integrated targeting vector. (C) PCR screening for homologous recombination of TRAF2 targeting vector in NIH3T3 cells (Lane 1: Molecular weight markers; Lane 2: full-length PCR product (predicted: 4600 bp), clone H23.3). Identity of the PCR product was verified by restriction digest of the product with BgIII (Lane 3: predicted fragments: 2580, 950, 600, 310, 184 bp); Lane 4: Molecular weight markers).

FIGS. 11A and 11B depict schematic designs of promoterless targeting constructs contained in targeting vectors.

### DETAILED DESCRIPTION OF THE INVENTION

Described herein is a new approach for studying the function of genes. The vectors and methods of the invention are generally applicable to a diverse range of cell types and genes. The invention provides vectors and methods of use thereof for targeted gene disruption in a somatic cell by a recombination event, for example, via homologous recombination. For example, the vectors of the present invention can be used to disrupt a gene of interest in a somatic cell line, e.g., a B cell line, a macrophage cell line, a fibroblast cell line, etc. The gene of interest may be one that is difficult or impossible to disrupt in a mouse, e.g., by knock-out methodology.

For purposes of the invention, a disrupted gene is one in which the function of one or more alleles of the gene have been altered by a recombination event, e.g., by homologous recombination. By “disrupted gene” is meant a portion of the genetic code has been altered, thereby affecting transcription and/or translation of that segment of the genetic code, e.g., rendering that segment of the code unreadable by insertion of an additional gene for a desired marker, e.g., a selectable marker, or by insertion of a regulatory sequence that modulates transcription of an existing sequence.

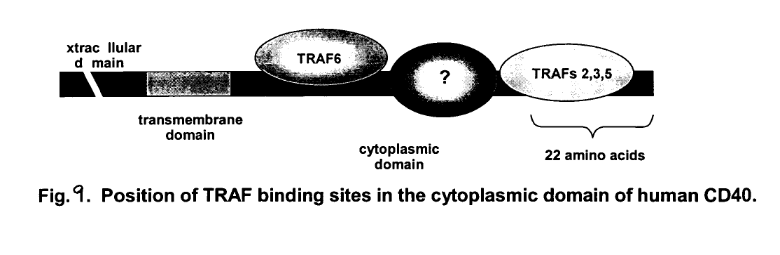

As an example, the vectors and methods described herein can be used for analysis of signaling molecule function, e.g., members of the TNFR-associated factor (TRAF) family, such as TRAF1, TRAF2, and TRAF3 (see, FIG. 9). In addition to TRAFs, there are numerous signaling molecules whose depletion in a whole animal, for example, by knock-out methodology, results in early lethality, or developmental defects so substantial that cells from these animals cannot be used to study normal cell function.

Using the system described herein to elucidate the roles of TRAF2 in B cell activation, it was discovered that TRAF2 participates in a variety of functions, in many of which it appears to share function with TRAF6. Results described herein also highlight nonredundant roles for TRAF2 in CD40-mediated IgM production and synergy with BCR signals.

I. Vectors of the Invention

The general methods for constructing vectors that can transform host cells are well known to those skilled in the art, and the same compositions and methods of construction may be utilized to produce the somatic cell gene targeting vectors described herein. For example, suitable methods of construction are disclosed in Sambrook and Russell, Molecular Cloning: A Laboratory Manual, 3rd ed., Cold Spring Harbor Laboratory Press, Cold Spring Harbor, N.Y. (2001).

The term “transformation” refers to the transfer of a nucleic acid fragment into the genome of a host cell, resulting in genetically stable inheritance. “Transformed,” “transgenic,” or “recombinant” refer to a host cell into which a heterologous nucleic acid molecule has been introduced through the transformation process. Nucleic acid molecules can be stably integrated into a host cell's genome using techniques generally known in the art (Sambrook and Russell, 2001). The term “untransformed” refers to normal cells that have not been through the transformation process.

“Chromosomally-integrated” refers to the integration of a foreign gene or DNA construct into the genomic DNA (i.e., genome) of the host cell by covalent bonds. “Genome” refers to the complete genetic material of an organism.

A “host cell” is a cell that has been transformed or a cell that is capable of transformation by an exogenous nucleic acid molecule. In particular, host cells of the present invention are somatic cells, e.g., a B cell or a macrophage. Where genes are not “chromosomally integrated” they may be “transiently expressed.” Transient expression of a gene refers to the expression of a gene that is not integrated into the host chromosome but functions independently, either as part of an autonomously replicating plasmid or expression cassette, for example, or as part of another biological system such as a virus.

A gene targeting vector of the invention can have a gene targeting construct, which can include, inter alia, cloning sites, DNA encoding a selectable marker and/or a polyadenylation sequence that are operably linked using techniques known to the art (Sambrook and Russell, 2001), as well as an expression cassette with a negatively selectable marker.

“Operably linked” nucleic acids are nucleic acids placed in a functional relationship with another nucleic acid sequence, e.g., DNA sequences linked on single nucleic acid fragment so that the function of one is affected by the other. For example, in the expression cassette of the invention, the functional linkage of a regulatory sequence, e.g., a promoter, is functionally linked to a heterologous nucleic acid sequence, e.g., DNA encoding a negatively selectable marker, resulting in expression of the latter. As another example, a promoter is operably linked to a coding sequence if it affects the transcription of the sequence. Generally, operably linked DNA sequences are DNA sequences that are contiguous. Linking is accomplished by ligation at convenient restriction sites. If such sites do not exist, synthetic oligonucleotide adaptors or linkers are used in accord with conventional practice.

“Promoter” refers to a nucleotide sequence, usually upstream (5′) to its coding sequence, which controls the expression of the coding sequence by providing the recognition for RNA polymerase and other factors required for proper transcription. “Promoter” includes a minimal promoter that is a short DNA sequence comprised of a TATA-box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers. Accordingly, an “enhancer” is a DNA sequence which can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. Both enhancers and other upstream promoter elements bind sequence-specific DNA-binding proteins that mediate their effects. Promoters may be derived in their entirety from a native gene, or be composed of different elements derived from different promoters found in nature, or even be comprised of synthetic DNA segments. A promoter may also contain DNA sequences that are involved in the binding of protein factors that control the effectiveness of transcription initiation in response to physiological or developmental conditions.

Generally, a vector of the present invention is a replicon, such as a plasmid, phage, virus, or cosmid, to which another DNA segment may be attached so as to bring about the replication of the attached segment. A “vector” is therefore, defined to include, inter alia, any plasmid, cosmid, phage or binary vector in double or single stranded linear or circular form which may or may not be self transmissible or mobilizable, and which can transform prokaryotic or eukaryotic host either by integration into the cellular genome or exist extrachromosomally (e.g., autonomous replicating plasmid with an origin of replication).

“Expression cassette” as used herein means a DNA sequence capable of directing expression of a particular nucleotide sequence in an appropriate host cell, comprising a promoter operably linked to the nucleotide sequence of interest that is operably linked to termination signals. It also typically includes sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components. The expression cassette may also be one that is naturally occurring but has been obtained in a recombinant form useful for heterologous expression. The expression of the nucleotide sequence in the expression cassette may be under the control of a constitutive promoter or of an inducible promoter that initiates transcription only when the host cell is exposed to some particular external stimulus. In the case of a multicellular organism, the promoter can also be specific to a particular tissue or organ or stage of development.

The term “gene” is used broadly to refer to any segment of nucleic acid associated with a biological function. Thus, genes include coding sequences and/or the regulatory sequences required for their expression. For example, gene refers to a nucleic acid fragment that expresses mRNA, functional RNA, or specific protein, including regulatory sequences. Genes also include nonexpressed DNA segments that, for example, form recognition sequences for other proteins. Genes can be obtained from a variety of sources, including cloning from a source of interest or synthesizing from known or predicted sequence information, and may include sequences designed to have desired parameters. In addition, a “gene” or a “recombinant gene” refers to a nucleic acid molecule comprising an open reading frame and including at least one exon and (optionally) an intron sequence. The term “introns” refers to a DNA sequence present in a given gene which is not translated into protein and is generally found between exons.

As used herein, “DNA” encompasses nucleic acids that are deoxyribonucleotides and polymers thereof in either single- or double-stranded form, composed of monomers (nucleotides) containing a sugar, phosphate and a base that is either a purine or pyrimidine. Unless specifically limited, the term DNA encompasses nucleotides containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated.

The terms “nucleic acid,” “nucleic acid molecule,” “nucleic acid fragment,” “nucleic acid sequence or segment,” or “polynucleotide” may also be used interchangeably with gene, cDNA, DNA and RNA encoded by a gene, e.g., genomic DNA, and even synthetic DNA sequences. The term also includes sequences that include any of the known base analogs of DNA and RNA.

The use of “promoterless” constructs for the gene targeting constructs of the the invention is desired. By “promoterless” is meant that the construct does not contain a promoter. As discussed herein, use of this feature makes expression of the constructs of the invention conditional upon homologous recombination at the targeted locus. The vector in which the construct is contained may have a promoter, but the promoter is not contained in the construct portion of the vector. Promoterless vectors are known in the art (see, for example, Sedivy and Dutriaux, 1999).

Selectable markers are utilized in the vectors of the invention to assay for the presence of the vector, and thus to confirm transfection. The presence of a positively selectable marker ensures the selection and growth of only those host cells, i.e., transfected somatic cells, which express the inserts. Typical positively selectable markers genes encode proteins that confer resistance to antibiotics and other toxic substances, e.g., histidinol, puromycin, hygromycin, neomycin, methotrexate, etc. It is preferred that a neomycin resistance gene, e.g., the gene encoding neomycin phosphotransferase, i.e., Neo^{R}, is used as the positively selectable marker.

Because inserting the selectable marker gene into the genome of the target somatic cell may have undesired positional effects on other genes of the somatic cell's chromosome, in one embodiment of the invention the selectable marker of the targeting construct is removable via a site-specific recombination event. Site-specific recombination occurs between specific, not necessarily homologous, pairs of sequences, and is enzyme mediated. For example, the intramolecular recombination that occurs between loxP sites is mediated by Cre recombinase. Site-specific recombination sequences are well known in the art, see for example the Cre-Lox system (U.S. Pat. No. 4,959,317) as well as the FLP/FRT site-specific recombination system (Lyznik et al., Nucleic Acids Res. 21(4):969-75 (1993)). It should be noted that a site-specific recombination event can be distinguished from a homologous recombination event. Homologous recombination occurs between homologous sequences of DNA, and is a rare event in somatic cells.

As discussed herein, the vectors of the invention may comprise a neomycin resistance gene as a selectable marker. It has been discovered that the neomycin resistance gene is a very effective mammalian drug resistance gene. Because in one embodiment a gene targeting vector of the invention can have a removable Neo^{R} gene, it may be used to target each allele of a gene of interest. In the past, scientists have tried to target a second allele by increasing the amount of drug added to the culture, reasoning that only cells in which both alleles containing, for example, a Neo^{R} gene could survive. However, experience with this method in germ cells has shown that unanticipated genetic changes can also be induced.

The gene targeting construct is flanked or positioned between two DNA segments, or “DNA flanks,” such that the construct can recombine with the gene of interest in the somatic cell by a recombination event. For example, the DNA flanks can be homologous to a genomic target, and the construct can recombine with the gene via homologous recombination. “Homology” refers to the percent identity between two polynucleotide or two polypeptide sequences. Two DNA or polypeptide sequences are “homologous” to each other when the sequences exhibit at least about 75% to 85%, preferably at least about 90%, and most preferably at least about 95% to 98% contiguous sequence identity over a defined length of the sequences. One DNA segment is homologous to a portion of genomic DNA that is 5′ (upstream) to the genomic target, and the other DNA segment is homologous to a portion of DNA that is 3′ (downstream) to the target. The DNA flanks are cloned into the construct using techniques known in the art. See, for example, Sambrook and Russell, 2001. Sources of the DNA flanks include DNA from any genomic source, as well as recombinant DNA sequences or segments.

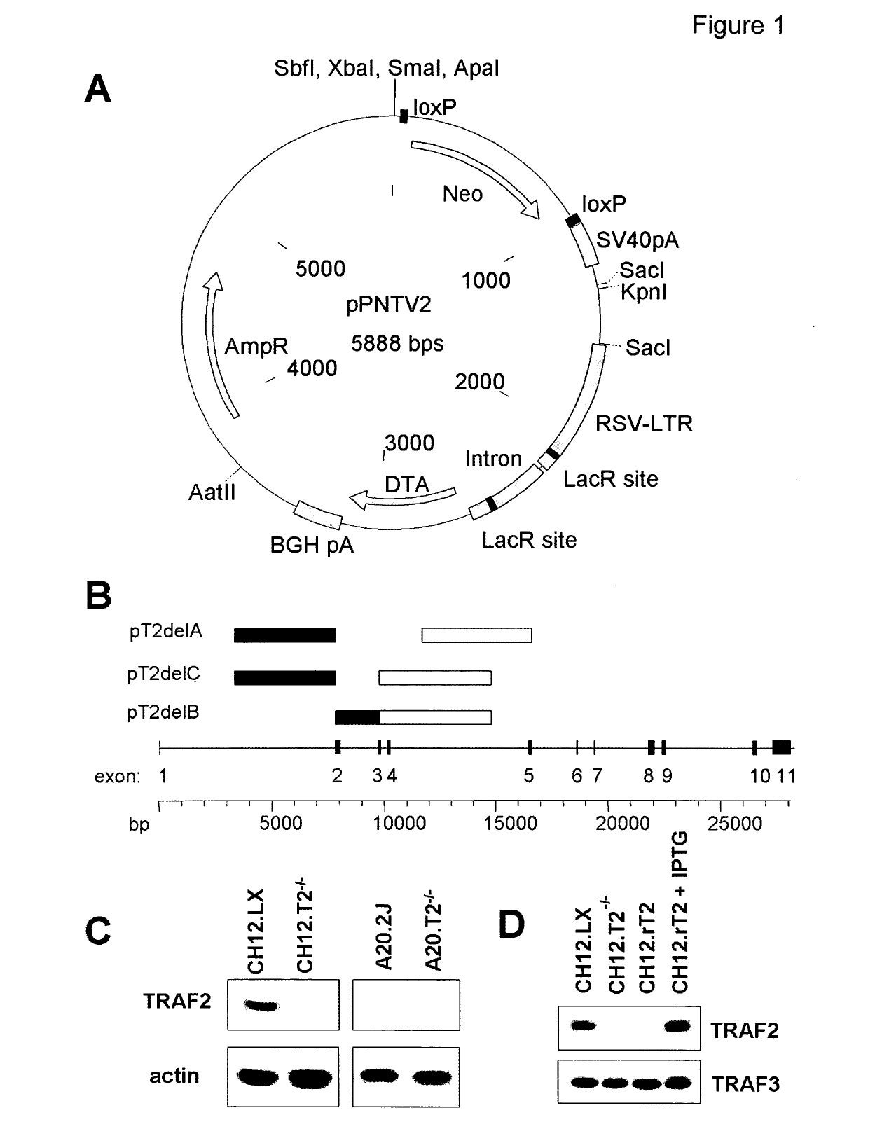

As described herein, the sequence of the DNA flanks can be based upon the sequence of a gene of interest, for example, the 5′ and 3′ flanks can be homologous to portions of a genomic sequence. The lengths of the DNA flanks will depend upon the uniqueness of the sequence to be targeted for recombination. For example, the flanks can be several thousand bases long. In certain embodiments, the flanks can be up to 5000 base pairs (bp) long, or up to 3000 bp long, or up to 2000 bp long, or up to 1000 bp long, or up to 800 bp long, or up to 600 bp long, or less. The space between the DNA flanks, i.e., the distance between the flanks in relation to the chromosomal target sequence, can vary (FIG. 1B). In various embodiments, the flanks may be designed to be less than about 1000 bp apart relative to the sequence of the genomic target, less than about 750 bp apart, less than about 500 bp apart, less than about 100 bp apart, less than about 50 bp apart, less than about 25 bp apart, less than about 10 bp apart, or less than about 5 bp.

The 5′ and 3′ DNA flanks are cloned into the construct such that they are operably linked with nucleic acid sequences present in the construct. As discussed herein, the construct is promoterless. Thus, when introduced into the targeted gene of the host cell, the DNA flanks of the construct can become operably linked to the promoter of the targeted gene. The expression of products encoded by the construct is thus made conditional upon homologous recombination at the targeted locus.

Although the vectors of the present invention are designed to strongly favor homologous recombination, non-homologous insertion of the gene targeting construct may occur. The use of a negatively selectable marker in the vector selects against recombination at the incorrect, i.e., nonhomologous, loci. A vector of the invention can incorporate a negatively selectable marker on an expression cassette that is downstream (3′) of the 3′ genomic DNA flank. Negatively selectable markers are known to the art and include, for example, the herpes simplex virus (HSV) thymidine kinase (TK) gene and the gene encoding diptheria toxin fragment A (DT-A). In one embodiment of the invention, the negatively selectable marker is DT-A.

If the negatively selectable marker gene encodes a toxin, such as DT-A, it can be placed behind a weak promoter, i.e., a promoter that controls expression of the toxin in such a manner as to prevent the toxin from killing cells before the gene targeting construct has had a chance to incorporate into the chromosome of the host somatic cell via homologous recombination. Examples of weak promoters are known to the art, and include, for example, a modified Rous sarcoma virus (RSV) promoter and the SV40 promoter. In particular, a modified RSV promoter may be used.

The term DNA “control elements” refers collectively to promoters, ribosome binding sites, polyadenylation signals, transcription termination sequences, upstream regulatory domains, enhancers, and the like, which collectively provide for the transcription and translation of a coding sequence in a host cell. Not all of these control sequences need always be present in a recombinant vector so long as the desired gene is capable of being transcribed and translated. Polyadenylation (“polyA”) sequences that can be used in the present vectors are known to the art, and include the SV40 polyA and the bovine growth hormone (BGH) polyA.

A control element, such as a promoter, “directs the transcription” of a coding sequence in a cell when RNA polymerase will bind the promoter and transcribe the coding sequence into mRNA, which is then translated into the polypeptide encoded by the coding sequence.

“Expression” refers to the transcription and/or translation of an endogenous gene or a transgene in cells.

A “nucleic acid fragment” is a fraction of a given nucleic acid molecule. Deoxyribonucleic acid (DNA) in the majority of organisms is the genetic material while ribonucleic acid (RNA) is involved in the transfer of information contained within DNA into proteins. The term “nucleotide sequence” refers to a polymer of DNA or RNA which can be single- or double-stranded, optionally containing synthetic, non-natural or altered nucleotide bases capable of incorporation into DNA or RNA polymers.

The terms “protein,” “peptide” and “polypeptide” are used interchangeably herein).

In the context of the present invention, an “isolated” or “purified” DNA molecule or an “isolated” or “purified” polypeptide is a DNA molecule or polypeptide that exists apart from its native environment and is therefore not a product of nature. An isolated DNA molecule or polypeptide may exist in a purified form or may exist in a non-native environment such as, for example, a transgenic host cell. For example, an “isolated” or “purified” nucleic acid molecule or protein, or biologically active portion thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein that is substantially free of cellular material includes preparations of protein or polypeptide having less than about 30%, 20%, 10%, 5%, (by dry weight) of contaminating protein. When the protein of the invention, or biologically active portion thereof, is recombinantly produced, preferably culture medium represents less than about 30%, 20%, 10%, or 5% (by dry weight) of chemical precursors or non-protein-of-interest chemicals. Fragments and variants of the disclosed nucleotide sequences and proteins or partial-length proteins encoded thereby are also encompassed by the present invention.

By “fragment,” or “portion” of a sequence is meant a full length or less than full length of the nucleotide sequence encoding, or the amino acid sequence of a polypeptide or protein. As it relates to a nucleic acid molecule, sequence or segment of the invention when linked to other sequences for expression, “portion” or “fragment,” means, for example, a sequence having at least 80 nucleotides, at least 150 nucleotides, or at least 400 nucleotides. If not employed for expressing, a “portion” or “fragment” means, for example, at least 9, at least 12, at least 15, or at least 20, consecutive nucleotides, e.g., probes and primers (oligonucleotides), corresponding to the nucleotide sequence of the nucleic acid molecules of the invention. Alternatively, fragments or portions of a nucleotide sequence that are useful as hybridization probes generally do not encode fragment proteins retaining biological activity. Thus, fragments or portions of a nucleotide sequence may range from at least about 6 nucleotides, about 9, about 12 nucleotides, about 20 nucleotides, about 50 nucleotides, about 100 nucleotides or more.

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of well-known molecular biology techniques, as, for example, with polymerase chain reaction (PCR) and hybridization techniques. Variant nucleotide sequences also include synthetically derived nucleotide sequences, such as those generated, for example, by using site-directed mutagenesis which encode the native protein, as well as those that encode a polypeptide having amino acid substitutions. Generally, nucleotide sequence variants of the invention will have at least 40, 50, 60, to 70%, e.g., preferably 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence.

“Recombinant” polypeptides refer to polypeptides produced by recombinant DNA techniques, i.e., produced from cells transformed by an exogenous DNA construct encoding the desired polypeptide. “Synthetic” polypeptides are those prepared by chemical synthesis. “Recombinant DNA molecule” is a combination of DNA sequences that are joined together using recombinant DNA technology and procedures used to join together DNA sequences as described, for example, in Sambrook and Russell, 2001.

The term “chimeric” refers to any gene or DNA that contains (1) DNA sequences, including regulatory and coding sequences, that are not found together in nature, or (2) sequences encoding parts of proteins not naturally adjoined, or (3) parts of promoters that are not naturally adjoined. Accordingly, a chimeric gene may comprise regulatory sequences and coding sequences that are derived from different sources, or comprise regulatory sequences and coding sequences derived from the same source, but arranged in a manner different from that found in nature.

A “transgene” refers to a gene that has been introduced into the genome by transformation and is stably maintained. Transgenes may include, for example, DNA that is either heterologous or homologous to the DNA of a particular cell to be transformed. Additionally, transgenes may comprise native genes inserted into a non-native organism, or chimeric genes. The term “endogenous gene” refers to a native gene in its natural location in the genome of an organism. A “foreign” gene refers to a gene not normally found in the host organism, but one that is introduced by gene transfer.

“Naturally occurring,” “native” or “wild type” is used to describe an object that can be found in nature as distinct from being artificially produced. For example, a protein or nucleotide sequence present in an organism (including a virus), that can be isolated from a source in nature and that has not been intentionally modified by a person in the laboratory is naturally occurring. Furthermore, “wild-type” refers to the normal gene, or organism found in nature without any known mutation, e.g., “native” or “wild type” proteins, polypeptides or peptides are proteins, polypeptides or peptides isolated from the source in which the proteins naturally occur.

“Coding sequence” refers to a DNA or RNA sequence that codes for a specific amino acid sequence and excludes the non-coding sequences. For example, a DNA “coding sequence” or a “sequence encoding” a particular polypeptide, is a DNA sequence that is transcribed and translated into a polypeptide in vitro or in vivo when placed under the control of appropriate regulatory elements. The boundaries of the coding sequence are determined by a start codon at the 5′-terminus and a translation stop codon at the 3′-terminus. A coding sequence can include, but is not limited to, procaryotic sequences, cDNA from eukaryotic mRNA, genomic DNA sequences from eukaryotic (e.g., mammalian) DNA, and even synthetic DNA sequences. A transcription termination sequence will usually be located 3′ to the coding sequence. It may constitute an “uninterrupted coding sequence,” i.e., lacking an intron, such as in a cDNA or it may include one or more introns bounded by appropriate splice junctions. An “intron” is a sequence of RNA that is contained in the primary transcript but is removed through cleavage and re-ligation of the RNA within the cell to create the mature mRNA that can be translated into a protein.

The terms “open reading flame” and “ORF” refer to the amino acid sequence encoded between translation initiation and termination codons of a coding sequence. The terms “initiation codon” and “termination codon” refer to a unit of three adjacent nucleotides (codon) in a coding sequence that specifies initiation and chain termination, respectively, of protein synthesis (mRNA translation).

“Regulatory sequences” and “suitable regulatory sequences” each refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include enhancers, promoters, translation leader sequences, introns, and polyadenylation signal sequences. They include natural and synthetic sequences as well as sequences that may be a combination of synthetic and natural sequences. As is noted above, the term “suitable regulatory sequences” is not limited to promoters. However, some suitable regulatory sequences useful in the present invention will include, but are not limited to constitutive promoters, tissue-specific promoters, development-specific promoters, inducible promoters and viral promoters.

“5′ non-coding sequence” refers to a nucleotide sequence located 5′ (upstream) to the coding sequence. It is present in the fully processed mRNA upstream of the initiation codon and may affect processing of the primary transcript to mRNA, mRNA stability or translation efficiency (Turner et al., 1995).

“3′ non-coding sequence” refers to nucleotide sequences located 3′ (downstream) to a coding sequence and include polyadenylation signal sequences and other sequences encoding regulatory signals capable of affecting mRNA processing or gene expression. The polyadenylation signal is usually characterized by affecting the addition of polyadenylic acid tracts to the 3′ end of the mRNA precursor.

The term “translation leader sequence” refers to that DNA sequence portion of a gene between the promoter and coding sequence that is transcribed into RNA and is present in the fully processed mRNA upstream (5′) of the translation start codon. The translation leader sequence may affect processing of the primary transcript to mRNA, mRNA stability or translation efficiency.

The term “mature” protein refers to a post-translationally processed polypeptide without its signal peptide. “Precursor” protein refers to the primary product of translation of an mRNA. “Signal peptide” refers to the amino terminal extension of a polypeptide, which is translated in conjunction with the polypeptide forming a precursor peptide and which is required for its entrance into the secretory pathway. The term “signal sequence” refers to a nucleotide sequence that encodes the signal peptide.

The “initiation site” is the position surrounding the first nucleotide that is part of the transcribed sequence, which is also defined as position+1. With respect to this site all other sequences of the gene and its controlling regions are numbered. Downstream sequences (i.e., further protein encoding sequences in the 3′ direction) are denominated positive, while upstream sequences (mostly of the controlling regions in the 5′ direction) are denominated negative.

Promoter elements, particularly a TATA element, that are inactive or that have greatly reduced promoter activity in the absence of upstream activation are referred to as “minimal or core promoters.” In the presence of a suitable transcription factor, the minimal promoter functions to permit transcription. A “minimal or core promoter” thus consists only of all basal elements needed for transcription initiation, e.g., a TATA box and/or an initiator.

“Transcription stop fragment” refers to nucleotide sequences that contain one or more regulatory signals, such as polyadenylation signal sequences, capable of terminating transcription. Examples include the 3′ non-regulatory regions of genes encoding nopaline synthase and the small subunit of ribulose bisphosphate carboxylase.

“Translation stop fragment” refers to nucleotide sequences that contain one or more regulatory signals, such as one or more termination codons in all three frames, capable of terminating translation. Insertion of a translation stop fragment adjacent to or near the initiation codon at the 5′ end of the coding sequence will result in no translation or improper translation. Excision of the translation stop fragment by site-specific recombination will leave a site-specific sequence in the coding sequence that does not interfere with proper translation using the initiation codon.

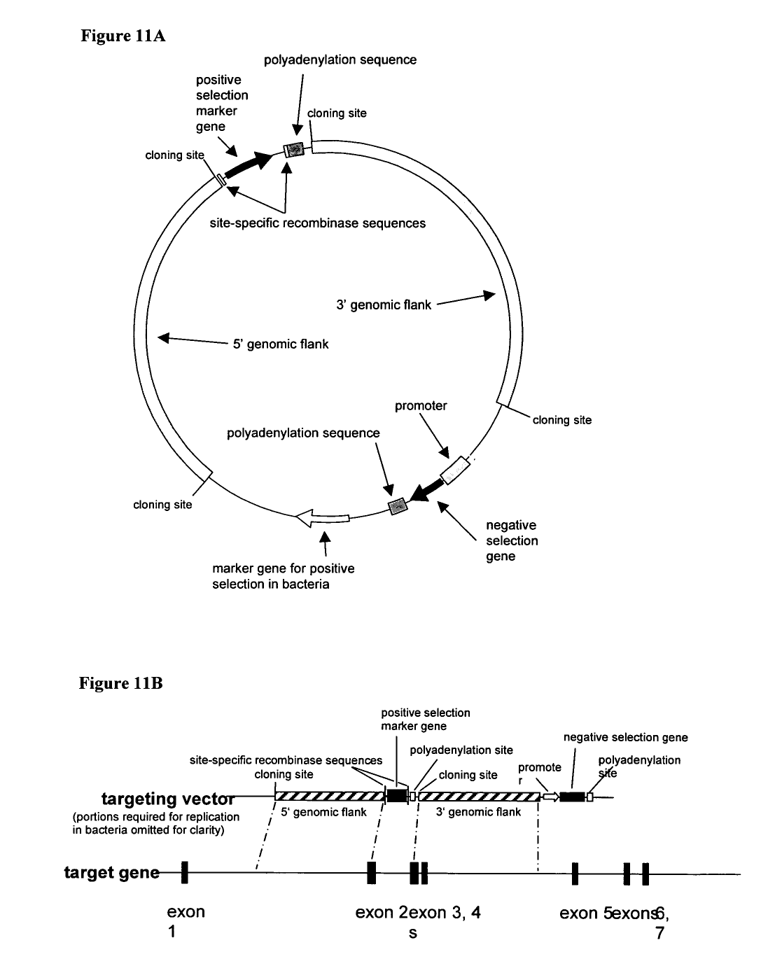

Embodiments of the invention can be found in FIGS. 11A and 11B.

II. Methods of the Invention

In the methods of the present invention, the promoterless gene targeting construct prevents expression of the positively selectable marker, located in the gene targeting construct, unless the construct recombines behind, i.e., downstream, of a promoter of the targeted gene of interest in the host somatic cell. The use of a promoterless construct therefore selects for recombination at the targeted locus. In this manner, expression of the selectable marker can be made conditional on homologous recombination at the target site. Homologous recombination in the somatic cell can be strongly favored. By favoring these recombination events, problems previously reported due to the low frequency of homologous recombination in somatic cells are overcome.

The gene targeting vector to be introduced into the somatic cells further will generally contain either a selectable marker gene or a reporter gene or both to facilitate identification and selection of transformed cells from the population of cells sought to be transformed. Both selectable markers and reporter genes may be flanked with appropriate regulatory sequences to enable expression in the host cells. Useful selectable markers are well known in the art and include, for example, antibiotic-resistance genes, such as Neo^{R} and the like.

Reporter genes are used for identifying potentially transformed cells and for evaluating the functionality of regulatory sequences. Reporter genes that encode for easily assayable proteins are well known in the art. In general, a reporter gene is a gene which is not present in or expressed by the recipient organism or tissue and which encodes a protein whose expression is manifested by some easily detectable property, e.g., enzymatic activity. Examples of reporter genes include the chloramphenicol acetyl transferase gene (cat) from Tn9 of E. coli and the luciferase gene from firefly Photinus pyralis. Expression of the reporter gene is assayed at a suitable time after the DNA has been introduced into the recipient cells.

If the gene targeting construct recombines with the targeted gene such that the 5′ DNA flank is inserted in frame with the gene's start codon, expression of a fusion protein comprising a portion of the targeted gene and the marker may result following homologous recombination. The term “fusion protein” is intended to describe at least two polypeptides, typically from different sources, which are operably linked. With regard to polypeptides, the term operably linked is intended to mean that the two polypeptides are connected in a manner such that each polypeptide can serve its intended function. Typically, the two polypeptides are covalently attached through peptide bonds. The fusion protein is preferably produced by standard recombinant DNA techniques. For example, a DNA molecule encoding the first polypeptide is ligated to another DNA molecule encoding the second polypeptide, and the resultant hybrid DNA molecule is expressed in a host cell to produce the fusion protein. The DNA molecules are ligated to each other in a 5′ to 3′ orientation such that, after ligation, the translational frame of the encoded polypeptides is not altered (i.e., the DNA molecules are ligated to each other in-frame).

Together with the polyadenylation sequence positioned downstream (3′) to the positively selectable marker gene on the gene targeting construct, the selectable markers of the targeting construct serve to disrupt transcription of the targeted gene in the host cell.

Targeted recombination of the gene targeting construct of the vector with a gene of interest will remove the negatively selectable marker. Non-targeted recombination events will leave the negatively selectable marker in place, and its expression will be toxic in the expressing cells, thus selecting against them.

The vectors can be readily introduced into somatic host cells, e.g., mammalian or insect cells, by transfection to yield a transformed cell having the vector stably integrated into its genome, so that the DNA molecules, sequences, or segments, of the vectors of the present invention are expressed by the host cell. The host cell may be a somatic cell of eukaryotic origin, e.g., mammalian or insect.

Physical methods to introduce a vector into a host cell include calcium phosphate precipitation, lipofection, particle bombardment, microinjection, electroporation, and the like.

In addition to transfecting a host cell with a vector of the invention, double-stranded oligonucleotides may be introduced into a somatic host cell. An “oligonucleotide,” as used herein, is a short polynucleotide or a portion of a polynucleotide. An oligonucleotide typically contains a sequence of less than about two hundred base pairs (bp). For example, in various embodiments, an oligonucleotide of the invention can contain about two hundred bp, at least 100 bp, at least 75 bp, at least 50 bp, or at least 20 bp.

In one embodiment, a oligonucleotide of 62 bp is introduced into a somatic host cell. This double-stranded DNA oligonucleotide is of random sequence. The addition of the oligonucleotide to the transfection mixture increases the frequency of homologous recombination. Transfections performed without the oligonucleotide, although resulting in neomycin-resistant clones, frequently yielded no homologous recombinants. With the oligonucleotide in the transfection mixture, the ratio of homologous recombination to random integration approached 1:10 in some cases. The oligonucleotide may serve as a sequence-independent decoy for cellular nucleases that would otherwise degrade or damage the targeting vector. It is also possible that the short oligonucleotide strands induce DNA repair enzymes that facilitate the process of homologous recombination.

To confirm the presence of the vector in the host cell, a variety of assays may be performed to detect a DNA sequence, e.g., a recombinant DNA sequence, of the vector. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

While Southern blotting and PCR may be used to detect the recombinant DNA segment in question, they do not provide information as to whether the DNA segment is being expressed. Expression may be evaluated by specifically identifying the peptide products of the introduced recombinant DNA sequences or evaluating the phenotypic changes brought about by the expression of the introduced recombinant DNA segment in the host cell, such as by antibiotic resistance.

As described herein, in one embodiment of the invention the selectable marker of the gene targeting construct is removable. The marker can be positioned between two site-specific recombination sequences, i.e., nucleotide sequences at which a recombinase can catalyze a site-specific recombination, such that the marker can be removed from a transfected cell by a site-specific recombination event. Contacting the transformed host cell with a recombinase will thus yield a genetically altered cell. “Genetically altered cells” denotes cells that have been modified by the introduction of recombinant or heterologous nucleic acids (e.g., one or more DNA constructs or their RNA counterparts) and further includes the progeny of such cells which retain part or all of such genetic modification. In one embodiment of the invention, the site-specific recombination sequences can be lox, e.g., loxP, sequences, and the recombinase can be Cre. Following selection for the positively selectable marker, e.g., neomycin resistance, the targeted somatic cell is transiently transfected with a plasmid encoding the bacterial recombinase Cre. Cre recognizes the loxP sites that flank the neomycin resistance gene and remove the neomycin resistance gene by a recombination event. One advantage of a removable positively selectable marker is that the targeted cell can be subsequently transfected with wild-type or mutant forms of the protein formally made by the disrupted gene of interests, using the positively selectable marker again for selecting re-transfected cells.

The invention will now be described by the following non-limiting examples.

### EXAMPLE 1

### Development and Use of TRAF-Deficient B Cell Lines

Introduction

CD40 is a member of the tumor necrosis factor receptor (TNFR) family, a group including a large number and variety of important immunoregulatory receptors. Engagement of CD40 by CD154 on activated T cells initiates signals that contribute to B cell proliferation, differentiation, isotype switching, antigen presentation, and other events necessary for an efficient humoral response (Foy et al., 1996). CD40 is also expressed on other antigen presenting cells such as macrophages and dendritic cells, and contributes to the activation of cell-mediated immunity (Stout and Suttles, 1996; Schoenberger et al., 1998; Bennett et al., 1998). Recently, CD40 has also been found on both CD4⁺ and CD8⁺ T cells, and has been posited to play an important potential role in both the development of normal T cell memory and autoimmunity (Bourgeois et al., 2002; Wagner et al., 2002).

In B lymphocytes, CD40 engagement results in the transcriptional upregulation of costimulatory molecules (CD80 and CD86), adhesion receptors (CD54, CD11a/CD18, CD23), and cytokines (IL-6 and TNF) (Bishop and Hostager, 2001). Increased expression of these proteins is partially attributed to activation of c-Jun NH₂-terminal kinase (JNK) and the transcription factor NF-κB. However, the mechanisms allowing CD40 to activate these factors remain unclear. Like other members of the TNFR family, signaling from CD40 involves proteins of the TNFR-associated factor (TRAF) family. This group of molecules serves as adapter proteins linking CD40 to downstream signaling events. TRAFs 2, 3, and 5 all bind to the membrane-distal CD40 cytoplasmic domain, while TRAF6 binds a membrane-proximal site. TRAFs 2-6 contain four major structural motifs. A carboxyl-terminal “TRAF-C” domain mediates binding to CD40 (Hu et al., 1994; Rothe et al. 1995; Ishida et al.,1996), while the neighboring “TRAF-N” domain contributes to interactions between TRAF molecules (Takeuchi et al., 1996). Near the amino terminus, TRAFs 2-6 contain a zinc RING motif and several zinc fingers. The zinc binding domains of TRAF2 participate in its ubiquitination when recruited to the CD40 signaling complex (Brown et al., 2002) and may interact with plasma membrane-associated molecules during CD40 signaling in B cells (Hostager et al., 2000).

In a variety of experiments, TRAF2 has been associated with the activation of JNK and NF-κB by TNFR family members (Inoue et al., 2000). Additional information concerning the role of TRAF2 in TNFR family signaling has been sought using TRAF2^{-/-} mice (Yeh et al., 1997). These experiments support a role for TRAF2 in JNK activation by TNF, as well as a contribution to TNF- or CD40-induced NF-κB activation. Unfortunately, as TRAF2^{-/-} mice die shortly after birth, more detailed analysis of CD40 signaling in their B cells has been difficult. The viability of the mice improves if produced on a TNF^{-/-} or TNFR1^{-/-} background, and B cells from such mice display defects in CD40-mediated NF-κB activation and proliferation (Nguyen et al., 1999). However, interpretation of these results is complicated by the fact that in normal B cells, CD40 stimulates the production of TNF, which in turn contributes to their activation (Hostager and Bishop 2002). It is also unclear if the activation defects in TRAF2^{-/-}/TNF^{-/-} (or TRAF2^{-/-}/TNFR1^{-/-}) B cells are directly related to the absence of TRAF2 in the CD40 signaling complex or if the combined deficiencies disrupt function of mature B cells in more indirect ways. An alternate method of assessing the contributions of TRAF molecules has been to examine the function of transgenic CD40 molecules with mutations in putative TRAF binding sites (Yasui et al., 2002; Jabara et al., 2002; Ahonen et al., 2002). However, levels of transgene expression and residual TRAF binding by CD40 mutants (Haxhinasto et al., 2002; Hostager and Bishop, 1999) may have contributed to differing conclusions amongst these studies.

To examine receptor signaling in the complete absence of individual or multiple TRAFs, and avoid the severe viability and development defects of TRAF^{-/-} mice, we have developed methodology to allow the efficient targeted disruption of TRAF (and other) genes in somatic cell lines. We have successfully applied this method to produce two mouse B cell lines specifically deficient in TRAF2. Using these B cells, in addition to their subclones stably expressing transfected wild-type (Wt) and mutant TRAF and CD40 molecules, we evaluated the contributions of TRAF2 to several CD40-mediated events not previously examined in TRAF1^{-/-} mice or mice bearing mutant CD40 transgenes. We found that the CD40-dependent degradation of TRAF3 is inhibited in cells lacking TRAF2, an observation relevant to the ubiquitination and degradation events recently found to be associated with TNFR family signaling (Brown et al., 2002; Li et al., 2002; Shi and Kehrl 2003; Deng et al., 2000). We also found that although some CD40 signals are TRAF2-independent, synergy between CD40 and the BCR in IgM production did not occur in TRAF2^{-/-} B cells. The TNF-dependent component of CD40-mediated IgM secretion was also found to be TRAF2-dependent, and CD40-mediated JNK activation was diminished in TRAF2^{-/-} B cells. Interestingly, we found that TRAF2 and TRAF6 make overlapping contributions to CD40-mediated NF-κB activation, reconciling and explaining the apparently disparate results of prior studies. These findings provide new information on the multiple roles played by TRAF2, using a novel approach applicable to the study of many other signaling receptors and pathways.

Experimental Procedures

Cell Lines—The mouse B lymphocyte line CH12.LX has been previously described (Bishop and Haughton, 1986). The diploid mouse B cell line A20.2J (Kim et al., 1979) was the gift of Dr. David McKean (Mayo Clinic, Rochester, Minn.). CH12.LX is diploid or near-diploid (karyotype analysis performed by Dr. Baoli Yang, University of Iowa). B cells were maintained in RPMI 1640 supplemented with 10% fetal calf serum, 10 μM 2-ME, and antibiotics. Sf9 insect cells were cultured in Grace's supplemented medium (Gibco, Grand Island, N.Y.) containing 10% FCS. High Five insect cells were grown in Express Five medium (Gibco).

CD154-Expressing Cells—Insect cells expressing mouse CD154 (mCD 154) were prepared as described (Hostager et al., 1996). A similar baculoviral expression construct was prepared for human CD154 (hCD154), using a commercially available kit (Clontech, Palo Alto, Calif.). Recombinant baculovirus and CD154-expressing insect cells (either Sf9 or High Five) were prepared by the Iowa Diabetes and Endocrinology Research Center (University of Iowa and VA Medical Center, Iowa City, Iowa). In all experiments using CD154-expressing cells, insect cells infected with wild-type baculovirus were used as negative controls. These cells were used in some experiments to again demonstrate that CD154 and anti-CD40 yield similar results in our assays, as shown in our previous reports (Brown et al., 2002; Hostager et al., 2000; Haxhinasto et al., 2002; Hostager et al., 1996).

Reagents and Materials—Proteinase K was from Roche Molecular Biochemicals (Indianapolis, Ind.). DNA oligonucleotide primers were obtained from IDT (Coralville, Iowa). Elongase DNA polymerase was from Invitrogen (Carlsbad, Calif.). G418 sulfate was from Gibco. Anti-TRAF2 antibody (Ab) used in Western blotting was from Medical and Biological Laboratories Co., LTD. (Nagoya, Japan). Western blotting Abs for cJun kinase, TRAF3, and TRAF6 were from Santa Cruz Biotechnology (Santa Cruz, Calif.). Sheep Ab used in human CD40 (hCD40) Western blots was described previously (Hostager et al., 2000). Abs for phospho-JNK, IκBα and phospho-IκBα were from Cell Signaling Technology (Beverly, Mass.). Anti-actin Ab was from Chemicon (Temecula, Calif.). mAbs against mCD40 (1C10(Heath et al., 1994), rat IgG2a), hCD40 (G28-5 (ATCC, Manassas Va.), mouse IgG1), and mouse IgE (EM-95.3 (Baniyash et al., 1984), rat IgG2a) were purified from hybridoma culture supernatants. Mouse IgG1 isotype control Ab (MOPC-21) and anti-FLAG Ab (M2) were from Sigma (St. Louis, Mo.). FITC-labeled anti-mCD80 and control Ab were from eBioscience (San Diego, Calif.). Hamster anti-mCD40, and control Ab were from BD Biosciences (San Jose, Calif.). HRP-labeled goat anti-rabbit and goat anti-mouse Abs were from BioRad (Hercules, Calif.), and HRP-labeled rabbit anti-sheep Ab was from Upstate (Waltham, Mass.). Recombinant mouse TNF was from R+D Systems (St. Paul, Minn.). Isopropyl-β-D-thiogalactopyranoside (IPTG) was from Amresco (Solon, Ohio). Cycloheximide was from Sigma. Protran nitrocellulose membrane (Schleicher and Schuell, Keene, N.H.) was used for JNK Western blots. Immobilon-P membrane (Millipore, Bedford, Mass.) was used for all remaining Western blots.

Preparation of Genomic DNA—Approximately 1×10⁵ cells were suspended in 25 μl digestion buffer (10 mM Tris, pH 8.0, 50 mM NaCl, 5 mM EDTA, 0.1% SDS) containing 60 μg/ml proteinase K (added immediately before use). The digests were incubated at 56° C. for 1 hr, then 10 min at 90° C. Genomic DNA templates were used at a final dilution of 1:100 in PCR.

PCR-PCR amplification of genomic DNA was performed with Elongase (Invitrogen, Carlsbad, Calif.), using the manufacturer's protocols.

Targeting Vectors—pUC19 was modified by the insertion of a promoterless neomycin resistance gene (flanked by loxP sites (Sauer and Henderson, 1989) and an SV40 polyadenylation (pA) site. Portions of these inserts were derived from p656 and IRES-neo-pA, provided by Dr. John Sedivy (Brown University, Providence R.I.). A diphtheria toxin-subunit A (DTA) gene was inserted into the targeting vector to select against cells carrying randomly inserted vector (Yanagawa et al., 1999). The toxin gene was positioned to be disrupted during the process of homologous recombination, but remain intact (and lethal) should the vector randomly integrate into the genome. Although a herpes simplex thymidine kinase cassette is often used for the same purpose, DTA proved more efficient in the B cell lines used and did not require the addition of gancyclovir to the culture medium. The diphtheria toxin cassette was provided by Drs. Matthew Anderson and Susumu Tonegawa (Massachusetts Institute of Technology, Cambridge, Mass.). Two versions of the basic targeting vector were produced, pPNTV1 (promoterless neomycin phosphotransferase targeting vector 1) and pPNTV2. In pPNTV1, diphtheria toxin (DTA) gene expression was driven by the phosphoglycerate kinase (PGK) promoter, and in pPNTV2 (FIG. 1A) by a weaker, modified RSV promoter from pOPRSV1.mcs1 (Busch and Bishop, 1999). To produce TRAF2-specific targeting vectors, genomic DNA sequences from the mouse TRAF2 gene (from the mouse B cell line M12.4.1 (Hamano et al., 1982) were inserted into endonuclease restriction sites flanking the neomycin resistance cassette in pPNTV1 or pPNTV2 . Three TRAF2-specific targeting vectors were produced, pT2delA, B and C. pPNTV1 was used for constructing pT2delA, and pPNTV2 was used for B and C. FIG. 1B illustrates the segments of the TRAF2 gene inserted into the three targeting vectors. PCR primers used in generating the 5′ genomic flank in pT2delA and C were pT2delA-5′F (tagtcgaccgtgaagtggtgctgatggt) (SEQ ID NO:1) and pT2delA-5′R (ctacccgggcagccatgtgagttacaacccccaca) (SEQ ID NO:2). PCR primers for the 3′ flank in pT2delA were T2delA-3′F (atggtaccgtctatgaaaggggccagtgtagt) (SEQ ID NO:3) and T2delA-3′R (aatctagaacagggtgctctgcagtg) (SEQ ID NO:4). Primers for the 5′ flank in pT2delB were T2delB-5′F (ttgtcgactgtgtgggggttgtaactcac) (SEQ ID NO:5) and T2delB-5′R (aatctagagtttaaactcagtcattcaatagacacaggcagc) (SEQ ID NO:6). Primers for the 3′ flank in pT2delB and pT2delC were T2delB-3′F (mggtacccagaactgtgctgcctgtgtc) (SEQ ID NO:7) and T2delB-3′R (aaggtaccaacagtttacagaaggtaagactaatg) (SEQ ID NO:8). In all targeting vectors, the 5′ genomic flanks were inserted so that the TRAF2 coding sequence was in frame with the neomycin phosphotransferase (NeoR) sequence. A promoterless NeoR cassette was used to reduce the number of antibiotic resistant clones resulting from random integration of the targeting constructs (Sedivy and Dutriaux, 1999). With homologous recombination, the endogenous TRAF2 promoter drives expression of a fusion protein consisting of a short segment of TRAF2 fused to NeoR. It has been shown that NeoR is particularly useful as an antibiotic resistance marker in promoterless gene targeting vectors, as even relatively weak genomic promoters drive sufficient expression to confer drug resistance (Hanson and Sedivy, 1995). The loxP sites flanking NeoR permit its removal after targeting each copy of the TRAF2 gene, and therefore allow neomycin selection to be used throughout the targeting process. Transient transfection of cells with a plasmid encoding Cre recombinase mediates recombination at the loxP sites and deletion of NeoR. Following the recombination event, the SV40 polyadenylation sequence remains in the TRAF2 gene to maintain disruption of expression. Removal of NeoR after the second round of targeting permits subsequent transfection of the cells with other neomycin-selectable expression plasmids.

TRAF2 and CD40 Vectors—IPTG-inducible TRAF2 and carboxyl-terminally 3XFLAG-tagged (Hernan et al., 2000) TRAF2 constructs were prepared using an expression system similar to that previously described (Hamano et al., 1982; Hsing and Bishop, 1999). The IPTG-inducible “dominant-negative” (DN) TRAF2 expression vector was previously described (Hostager and Bishop, 1999). Using PCR-based mutagenesis, expression vectors encoding hybrid CD40 molecules were prepared. One hybrid consists of the extracellular domain of hCD40 fused to the transmembrane and cytoplasmic (CY) domains of mCD40 (hmCD40). The second hybrid, hmCD40ΔT6, contains point mutations at two residues in the putative TRAF6 binding site (Manning et al., 2002). Both constructs were inserted into the pRSV.5(neo) expression vector (Long et al., 1991). An expression vector encoding hCD40Δ22 was previously described (Hostager et al., 1996). Stable transfection of cells with CD40 and TRAF2 constructs by electroporation was performed as described below.

Transfection of Cells With Targeting Vectors—Cells (1.5×10⁷) were suspended in 400 μl RPMI supplemented with 1.54 mg/ml glutathione, 5 μg linearized targeting vector, and 5 μg double stranded DNA oligonucleotide of random sequence (62 bp; synthesized by IDT). The addition of the oligonucleotide to the transfection mixture appeared to increase the frequency of homologous recombination. Transfections performed without the oligonucleotide, while resulting in neomycin-resistant clones, frequently yielded no homologous recombinants. With the oligonucleotide in the transfection mixture, the ratio of homologous recombination to random integration approached 1:10 in some cases. We speculate that the oligonucleotide may serve as a sequence-independent decoy for cellular nucleases that would otherwise degrade or damage the targeting vector. It is also possible that the short oligonucleotide strands induce DNA repair enzymes that facilitate the process of homologous recombination. Cells were electroporated using an ECM 830 electroporator (Genetronics, San Diego, Calif.). Settings for CH12.LX cells were 200 V/30 ms, and for A20.2J cells, 225 V/30 ms (4 mm gap cuvettes). After electroporation, cells were placed on ice for 5-10 minutes, then diluted in 10 ml culture medium supplemented with 15% FCS and cultured overnight. Cells were subcloned in medium containing 400 μg G418 sulfate. Approximately 10-14 days after electroporation, neomycin-resistant clones were screened for homologous recombination.

Screening for Homologous Recombination—A PCR-based assay was used to screen clones for homologous recombination. Screening of pT2delA and C transfectants was performed with the PCR primers T2delA-5′S (cttagttttcacaatgccttcg) (SEQ ID NO:9) and rNeo (caatccatcttgttcagccat) (SEQ ID NO:10). T2delA-5 ′S is complementary to genomic sequence immediately upstream of the sequence used as the 5′ flank in the targeting vectors, and the 3′ primer is complementary to a portion of NeoR. PCR amplification of genomic DNA from homologous recombinants produced a product of approximately 4500 bp (not shown). As positive controls, several genomic DNA samples from each transfection were PCR amplified with T2delA-5′S and the T2delA-5′R. PCR screening of pT2delB transfectants was accomplished using rNeo and T2delB-5′S (gaattgaggtgtgatatggtctgtg) (SEQ ID NO:11). PCR amplification of genomic DNA from homologous recombinants produced a product of approximately 2000 bp. In positive control reactions, T2delB-5′S and T2delB-5′R were used.

Removal of NeoR—To mediate recombination at the loxP sequences, cells were transiently transfected with pBS185, coding for Cre recombinase (Sauer, 1993). 1×10⁷ cells were suspended in 400 μl RPMI containing 1.54 mg/ml glutathione and 15 μg pBS185. Cells were electroporated as above, then subcloned in medium without G418. After approximately 10 days of culture, clones were tested for G418 sensitivity. Typically, 5-10% of the clones were G418-sensitive.

TRAF3 Degradation Assay—Cells (5×10⁶) were stimulated for six hours in a volume of 0.5 ml at 37° C. with 10 μg/ml hamster anti-mCD40 or an isotype control Ab. Where indicated, new protein synthesis was blocked by adding 1.0 μM cycloheximide to the cell cultures 30 minutes prior to the addition of the stimulatory antibody (Catlett et al., 2001). The cells were then pelleted by centrifugation, and whole-cell lysates prepared by resuspending the cells in 200 μl 2×SDS PAGE loading buffer and sonicating briefly. 2.5×10⁵ cell equivalents were loaded per lane on SDS PAGE gels. TRAF3 was quantitated on Western blots using a low-light imaging system (LAS-1000, FUJIFILM Medical Systems USA, Inc., Stamford, Conn.). Western blots were simultaneously probed for actin, which allowed for normalization of the TRAF3 signal in each lane.

JNK Assay—Activated JNK was detected on Western blots using a polyclonal antiserum specific for JNK phosphorylated on Thr¹⁸³ and Tyr¹⁸⁵. Briefly, 1×10⁶ B cells were assayed per stimulation condition. Cells were stimulated for various times in a volume of 1 ml at 37° C. with 5 μg/ml hamster anti-mCD40 or an isotype control Ab. Following stimulation, cells were pelleted by centrifugation, and whole-cell lysates prepared as in TRAF3 degradation experiments. 1×10⁵ cell equivalents were loaded per lane on SDS PAGE gels. Proteins were transferred to nitrocellulose for Western blotting with antibodies for anti-phospho-JNK and total JNK.

NFκB Activation Assay—NFκB activation was measured by the phosphorylation and degradation of IκBα appearing on Western blots of whole cell lysates, using anti-phospho-IκBα and anti-IκBα Abs according to manufacturer's instructions. 1×10⁶ cells were stimulated at 37° C. in a volume of 1 ml, using 5 μg/ml anti-CD40 mAbs (1C10 and G28-5) or isotype control mAbs. Whole cell lysates were prepared as in JNK assays.

IgM Secretion Assay—Quantitation of IgM secreting CH12.LX cells was accomplished as described (Bishop, 1991). Briefly, B cells were incubated with various stimuli for 72 hrs., viable cells counted by trypan blue exclusion, mixed with sheep erythrocytes (SRBC) and guinea pig complement, and transferred to chamber slides. Slides were incubated for 30 min at 37° C. Activated CH12.LX cells secrete IgM specific for phosphatidyl choline present on SRBC, and create lytic plaques on a lawn of SRBC in the presence of complement. In experiments with cells expressing IPTG-inducible TRAF2, cells were incubated for 24 hrs with 100 μM IPTG, then stimulated for 48 hrs. Stimuli used were anti-CD40 (1C10 or G28-5, 2 μg/ml), mouse- or human CD154-expressing insect cells (1 insect cell per 10 B cells), SRBC (antigen, 0.1%), and recombinant mouse TNF (50 pg/ml). Results are presented as the ratio of plaque-forming cells (Pfc) to viable recovered cells.

Immunoprecipitation—For examining TRAF-CD40 interactions by coimmunoprecipitation, cells (2×10⁷) were stimulated for 20 minutes (37° C.) with 1×10⁶ High Five cells infected with wild-type baculovirus or baculovirus encoding hCD154. Cells were lysed and hCD40 was immunoprecipitated from membrane microdomains as described (Busch and Bishop, 2001).

CD80 Upregulation—Cells were stimulated for 72 hr. with 5 μg/ml anti-mCD40 (1C10), anti-hCD40, or appropriate isotype controls and assayed by flow cytometry as described previously (Hostager et al., 1996).

Flow Cytometry—Staining of cells for flow cytometry was described (Hostager et al., 1996). Cells were analyzed with a FACScan flow cytometer (BD Biosciences), and WinMDI software (The Scripps Research Institute, San Diego, Calif.).

Results

Generation of TRAF2^{-/-} Cells by Homologous Recombination—To generate TRAF2^{-/-} B cell lines, we constructed targeting vectors containing segments of the mouse TRAF2 gene interrupted by neomycin resistance cassettes. The DNA constructs were designed to undergo homologous recombination with the TRAF2 genes in cells, disrupting TRAF2 production. Although disruption of genes by homologous recombination has been very successful in murine embryonic stem cells, it has been used -infrequently in somatic cell lines due to the often abysmal ratio of homologous to non-homologous recombination events (Sedivy and Dutriaux, 1999). We used a number of strategies to improve the frequency of homologous recombination so this technique could be used to generate somatic cell lines deficient in specific genes in a timely fashion with reasonable effort. Details of the technique are presented in Experimental Procedures; the basic design of the targeting constructs is shown in FIG. 1.

Three targeting constructs were produced for disrupting the TRAF2 gene in CH12.LX and A20.2J B cells. These cell lines were chosen because they are diploid and have been used in many studies by multiple investigators as valid models of B cell activation events, including those mediated by CD40. The regions of genomic DNA used in each of the targeting constructs are shown in FIG. 1B. In CH12.LX cells, pT2delA then pT2delB were used to sequentially target the two TRAF2 genes. Much of the genomic sequence in pT2delB was derived from regions of the TRAF2 gene deleted in the first round of targeting, eliminating retargeting of the pT2delA-targeted gene in the second round. A similar approach was used in A20.2J cells, although to increase the frequency of homologous recombination, the vector used in the first round of targeting (pT2de1C) was designed to produce a smaller deletion in the genomic sequence than did pT2delA. In the second round of targeting, pT2delB was again used. PCR was used to screen for homologous recombination after each round of targeting, and to confirm removal of NeoR. In TRAF2-deficient CH12.LX cells (CH12.T2^{-/-}), RT PCR for TRAF2 mRNA revealed the presence of a defective transcript arising from the pT2delB-targeted copy of TRAF2. In this defective transcript, mRNA splicing removed the SV40 pA signal sequence and generated a frameshift between the upstream and downstream sequence (data not shown). Western blots of whole-cell lysates confirmed disruption of TRAF2 protein expression (FIG. 1C). CH12.T2^{-/-} cells stably transfected with an IPTG-inducible TRAF2 expression plasmid (CH12.rT2) served as controls in several experiments. Levels of TRAF2 expression in the presence and absence of inducer are illustrated in FIG. 1D.

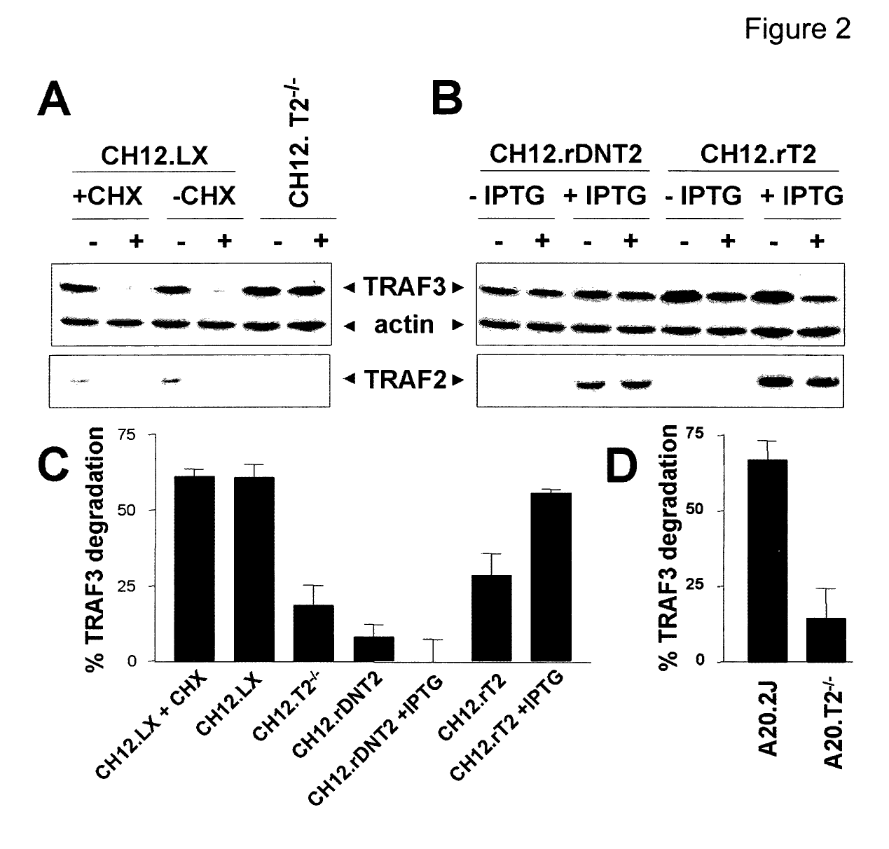

Defective TRAF3 Degradation in TRAF2-Deficient Cells—Our recent work has indicated that ubiquitination and degradation events are coupled with signaling through CD40. Specifically, we demonstrated that TRAF2 is ubiquitinated and degraded as a result of CD40 signaling (Brown et al., 2002); this process appears to play an important role in normal regulation of the duration and strength of CD40 signaling (Brown et al., 2001). These events can be disrupted by mutations in the zinc RING motif present in the amino terminal domain of TRAF2. A recent report indicates that TRAF2 can promote its own ubiquitination in response to TNF receptor signaling (Shi and Kehrl, 2003). While the ubiquitination events associated with signaling may contribute to negative regulation of signaling (Brown et al., 2002), these events may also be integral to the activation of certain signaling pathways (Shi and Kehrl, 2003; Wang et al., 2001). In previous work, we observed CD40-induced modification (Hostager et al., 2000) and degradation (Brown, et al., 2001) of TRAF3. Interestingly, these events were not observed as a result of signaling through LMP1, a viral CD40 mimic that interacts strongly with TRAF3, but only weakly with TRAF2 (Brown, et al., 2001). We therefore tested the possibility that TRAF2 participates in the CD40-induced degradation of TRAF3, using our TRAF2-deficient cell lines. Stimulation of CH 12.LX cells with anti-CD40 mAb for six hours resulted in ˜63% reduction of the amount of TRAF3 detected by Western blot, while the reduction of TRAF3 levels in CH12.T2^{-/-} cells was only ˜13% (FIG. 2). A similar defect in TRAF3 degradation was observed in TRAF2-deficient A20.2J cells. New protein synthesis was not required for CD40-induced TRAF3 degradation, indicating that the degradation does not occur via induction of other TNFR family receptors or their ligands (FIG. 2). In CH 12.T2^{-/-} cells reconstituted with IPTG-inducible TRAF2, even low level TRAF2 expression occurring in the absence of IPTG (FIG. 1D) partially reversed the defect in degradation (FIG. 2). IPTG induction of TRAF2 expression to normal endogenous levels increased CD40-mediated TRAF3 degradation. Expression of a TRAF2 mutant lacking its amino terminal RING motif, previously shown to have a “dominant-negative” (DN) effect on various CD40 functions (Rothe et al., 1995; Hostager and Bishop, 1999), failed to restore TRAF3 degradation, indicating the importance of the TRAF2 RING in this function. That a minor amount of TRAF3 degradation occurs in the absence of TRAF2 suggests that other CD40-associated molecules may make small contributions to degradation. Together, these results indicate that TRAF2 plays an important role in the activation-induced degradation of TRAF3, and that the RING motif of TRAF2 is required.

Multiple Roles for TRAF2 in Regulating IgM Production—Previously, we demonstrated that stimulation through CD40 results in the activation of IgM secretion through at least two signaling pathways (Hostager and Bishop, 2002; Hostager and Bishop 1999). The first, directly linked to CD40, is TRAF2-independent as it is activated by CD40 mutants unable to bind TRAF2 (Haxhinasto et al., 2002; Hostager and Bishop, 1999; Hostager et al., 1996). The second pathway is dependent upon CD40-induced TNF, acting in an autocrine fashion through CD120b and TRAF2 to augment IgM secretion (Hostager and Bishop, 2002).

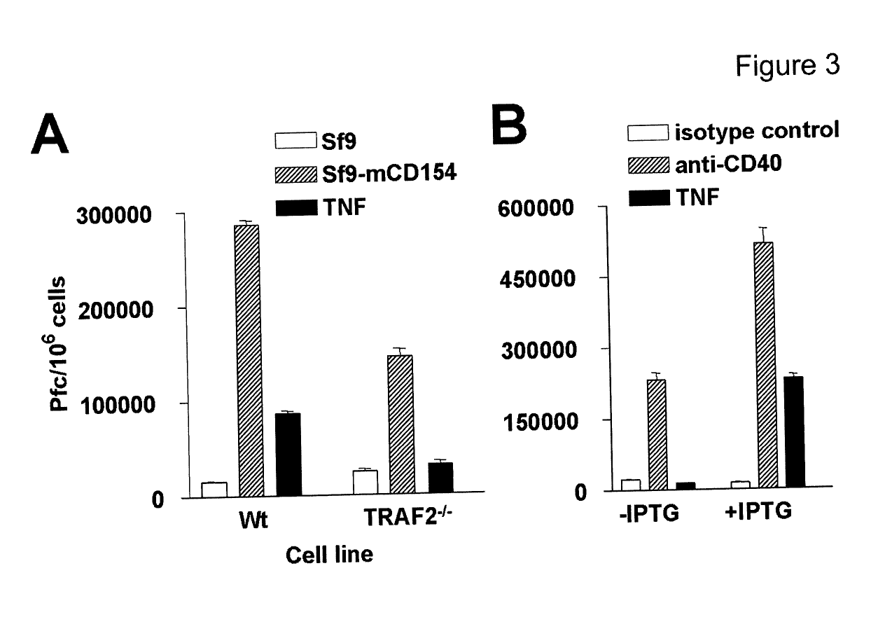

Consistent with this model, virtually no CH12.T2^{-/-} cells could be activated to secrete IgM in response to stimulation by TNF (FIG. 3A). CH12.T2^{-/-} cells also appeared to have a decreased response to CD40 stimulation. However, it is important to note that absolute responses among different CH12.LX subclones often vary in this assay, and could account for the apparent decrease in IgM secretion. To ensure that the observed defects were due to the lack of TRAF2 and not simply variation among clones, we examined IgM secretion by CH12.T2^{-/-} cells reconstituted with IPTG-inducible TRAF2 (FIG. 3B). In the presence of IPTG, responses to CD40 were enhanced and the TNF response was fully restored, indicating that TRAF2 contributes to both responses. Similar results were obtained regardless of whether mCD 154 (FIG. 3A) or anti-mCD40 (FIG. 3B) was used to activate CD40 signaling.

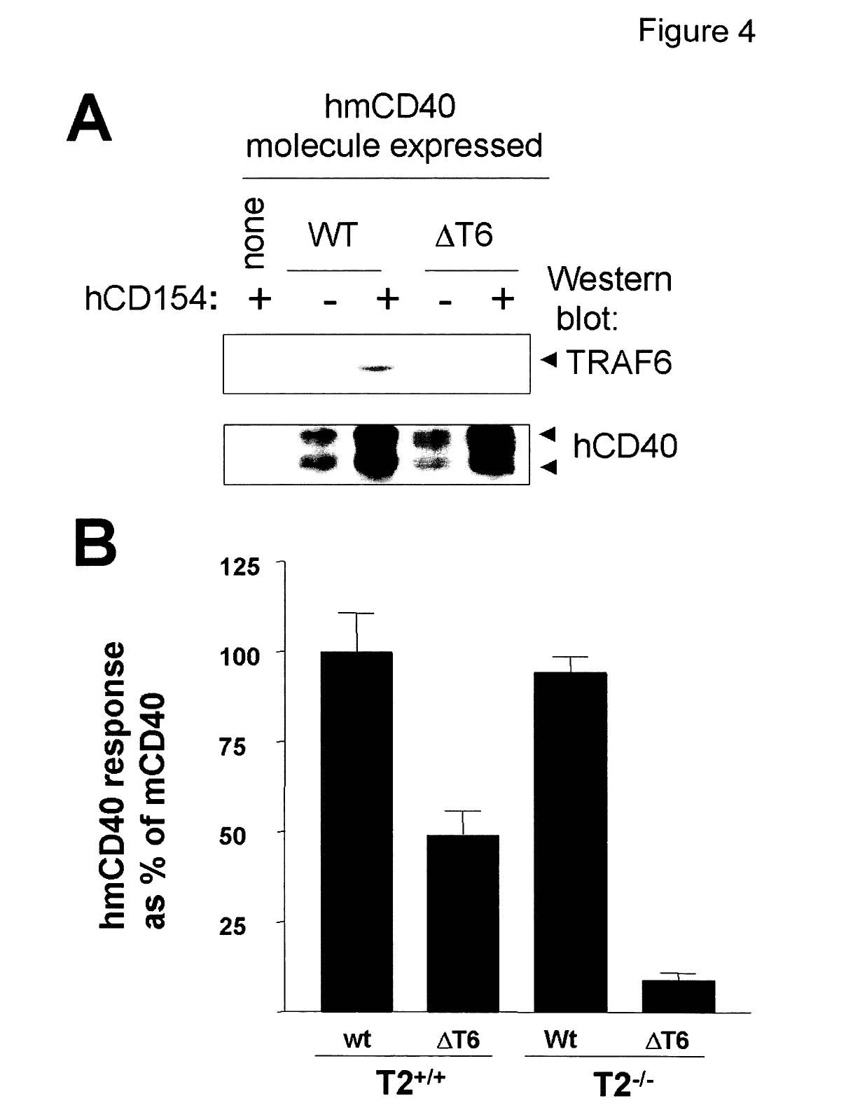

While TNF-induced IgM secretion is highly dependent on TRAF2, other molecules must also contribute to the activation of IgM secretion by CD40. TRAF2-deficient CH12.LX cells allowed us to examine potential contributions of TRAF6 to CD40-mediated IgM secretion in the absence of TNF-induced signaling, which has not been possible in other model systems. Previous studies indicate that the TRAF6 binding site in the cytoplasmic (CY) domain of CD40 can be disrupted by minor modifications in amino acid sequence (two amino acid changes) (Manning et al., 2002; Jalukar et al., 2000). We generated an expression construct encoding a hybrid CD40 molecule consisting of the extracellular domain of hCD40 fused to the transmembrane and CY domains of mCD40 containing the appropriate mutations (hmCD40ΔT6). A similar construct having a wild-type CY domain was also prepared. The hybrid molecules were then transfected and stably expressed in CH12.LX and its TRAF2-deficient counterpart (equivalent hCD40 expression was determined by flow cytometry, not shown). The extracellular domain of hCD40 was used in the mutant construct to allow differential stimulation of the cells through the transfected mutant and their endogenous (wild-type) mCD40. Mouse and human CD40 are sufficiently different that non-cross-reacting agonistic mAbs for the two molecules are available. Alternatively, hCD154 can be used to engage the hybrid molecule, and has virtually no capacity to stimulate cells through mCD40. To determine if the mutant CY domain of the hybrid was defective in TRAF6 binding in mouse B lymphocytes, the transfected cell lines were first stimulated through the hCD40 extracellular domain (to induce TRAF recruitment), then cell lysates were prepared from which the hybrid molecules were immunopreciptiated. Western blot analysis of the immunoprecipitates revealed that the hmCD40ΔT6 was deficient in TRAF6 binding (FIG. 4A). In CH12.LX, both hybrid molecules were able to stimulate IgM secretion, with hmCD40ΔT6 displaying a partial defect when compared to endogenous mCD40 (FIG. 4B), consistent with our previous results using hCD40 molecules defective in TRAF6 binding (Jalukar et al., 2000). As noted earlier, it is not possible to compare the absolute amounts of IgM secretion of two different clones, and we therefore used endogenous mCD40 activation of each individual clone as the basis for comparison. In TRAF2^{-/-1} cells, hmCD40ΔT6 displayed a greater defect in stimulation of IgM secretion than it did in the parental cell line, indicating that both TRAF2 and the TRAF6 binding site make unique as well as cooperative contributions to CD40-mediated IgM production.

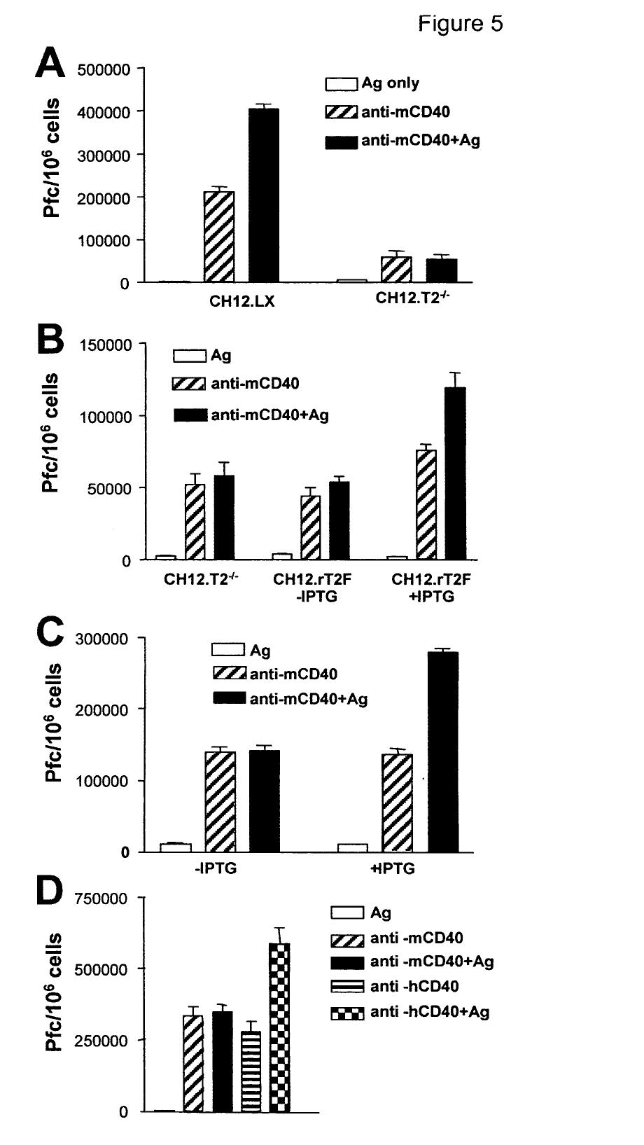

We previously demonstrated that BCR signals, while unable to stimulate IgM secretion in CH12.LX cells, markedly enhance CD40-mediated IgM secretion (Bishop et al., 1995). Enhancement of CD40-mediated activation by BCR signals also occurs in splenic B cells (Haxhinasto et al., 2002). This cooperation is evident even in cells stimulated though hCD40Δ22 (Hostager et al., 1996), a mutant having a truncated CY domain rendering it incapable of binding either TRAF2 or TRAF3 (Haxhinasto et al., 2002; Hostager and Bishop, 1999). We therefore concluded that TRAF2 is not directly required for the synergy between the BCR and CD40. However, our recent work indicates that TRAF2 contributes to BCR-CD40 synergy, possibly by blocking an inhibitory effect of TRAF3 or an unknown molecule that binds to the same region of CD40 as TRAF3 (Haxhinasto et al., 2002). Our TRAF2^{-/-} B cells allowed us to test this possibility directly. Little if any synergy between the BCR and CD40 was observed in TRAF2^{-/-} cells, supporting the hypothesis that TRAF2 makes a crucial contribution to the cooperation (FIG. 5A). Reconstitution of TRAF2 expression in the deficient cells restored cooperation between the BCR and CD40 (FIG. 5B). Surprisingly, synergy between the BCR and CD40 was also restored in TRAF2^{-/-} cells transfected with DNTRAF2 (FIG. 5C). As shown in FIG. 2, this mutant failed to restore the CD40-induced degradation of TRAF3, suggesting that TRAF3 degradation is not critical for the synergy of CD40 and BCR signals. This supports our hypothesis that the major role of TRAF2 in BCR-CD40 synergy is to prevent the binding of an inhibitory factor to the CY domain of CD40. To examine this possibility further, we transfected the TRAF2-deficient cells with hCD40Δ22 and stimulated the cells in the presence or absence of BCR engagement. Synergy was evident when stimulating through hCD40Δ22, but not when cells were stimulated through endogenous Wt CD40 (FIG. 5D). These results are consistent with the concept that TRAF2 blocks the binding of an inhibitory molecule to the CY domain of Wt CD40, and that this molecule, like TRAF2, cannot bind hCD40Δ22. A likely candidate is TRAF3. This is a previously unappreciated and novel role for TRAF2.

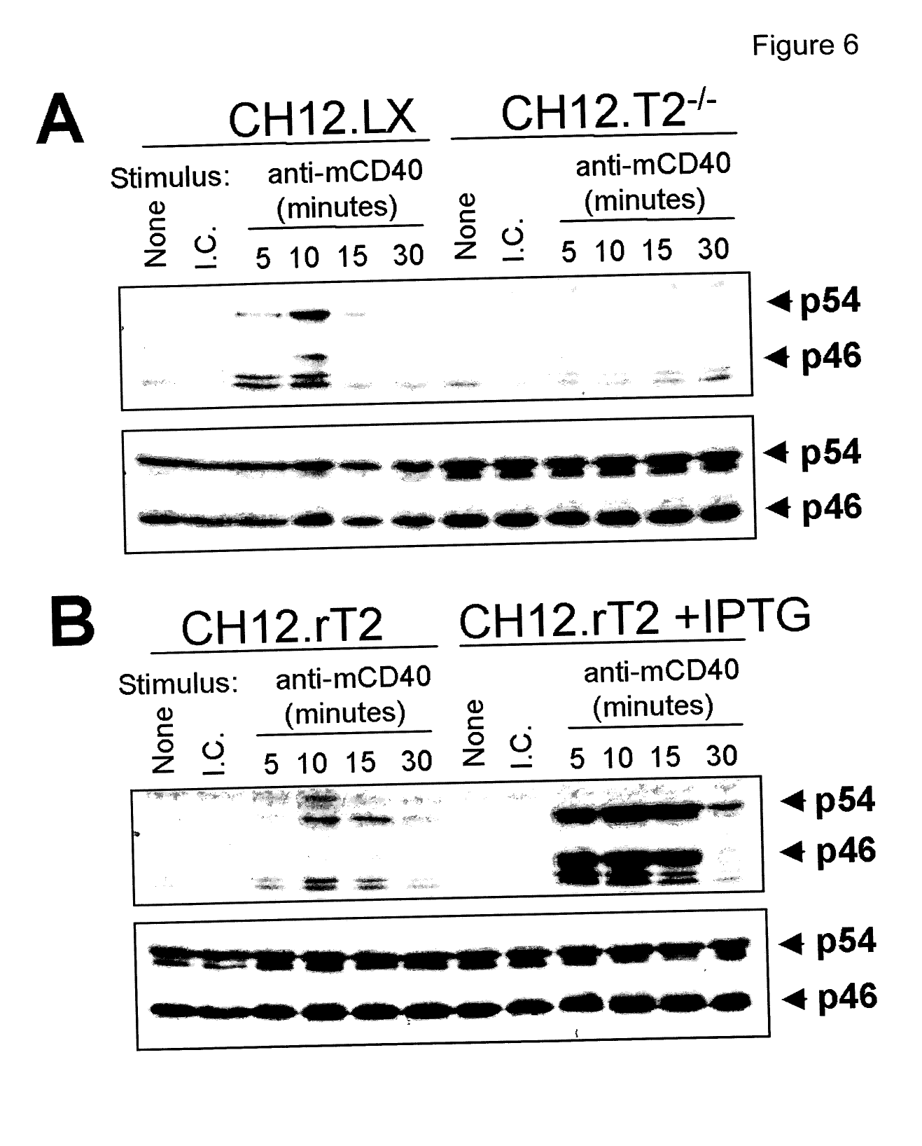

TRAF2 is Essential for Optimal JNK Activation—Previous work with TRAF2^{-/-} embryonic fibroblasts (Yeh et al., 1997) showed that these cells are defective in TNF-mediated JNK activation. However, different TNFR family members and different cell types may utilize TRAFs differently, and CD40-mediated JNK activation in TRAF2-deficient cells has not been examined. Data from transgenic B cells expressing a dominant-negative TRAF2 (Lee et al., 1997) suggest that TRAF2 contributes to the activation of JNK by CD40 and other TNFR family members. However, excess mutant TRAF2 is likely to have effects in addition to blocking the binding of normal TRAF2, especially in the case of CD40 where a number of other TRAFs (e.g. TRAFs 1, 3, and 5) bind a site that overlaps with the TRAF2 binding site. Thus, there are complexities in data interpretation using DNTRAF2 molecules. Using our TRAF2^{-/-} B cells, we found that CD40-mediated JNK activation (FIG. 6A) is markedly defective. To ensure that the defect in JNK activation was due to the disruption of TRAF2 expression, parallel experiments were performed using TRAF2^{-/-} CH12.LX cells reconstituted with IPTG-inducible TRAF2. In the absence of IPTG, a small amount of TRAF2 was expressed in the cell line (FIG. 1D), and partially restored the response to anti-CD40 mAb (FIG. 6B). Induction of TRAF2 with IPTG resulted in expression levels greater than in parental CH12.LX cells (FIG. 1D), and resulted in an enhanced JNK response (FIG. 6B). Taken together, these data establish TRAF2 as a major contributor to CD40-mediated JNK activation in B cells.

TRAF2 and TRAF6 Make Overlapping Contributions to NF-κB Activation and CD80 Upregulation—The role of TRAF2 in activation of NF-κB by TNFR family members has been particularly confusing. Early studies in which TRAF2 or DNTRAF2 were overexpressed in epithelial cells suggested that TRAF2 is essential to this function(Rothe et al., 1995). However, we showed that NF-κB activation in B cells by CD40 molecules with defective TRAF2 binding is only slightly lower than that stimulated by WtCD40 (Hsing et al., 1997). Similarly, in murine TRAF2-deficient embryonic fibroblasts, TNF-mediated NF-κB activation is only slightly slower than that observed in normal cells, suggesting that TRAF2 plays a minor role in the activation of this signaling pathway by TNFR family members (Yeh et al., 1997). TRAFs 5 and 6 have also been implicated as inducing NF-κB activation in overexpression studies (Ishida et al., 1996; Ishida et al., 1996; Nakano et al., 1996; Cao et al., 1996), but CD40-mediated NF-κB activation appears normal in TRAF5-deficient mice (Nakano et la., 1999) and we find that CD40 molecules that do not bind detectably to TRAF6 activate NF-κB normally in B cells (Julukar et al., 2000).

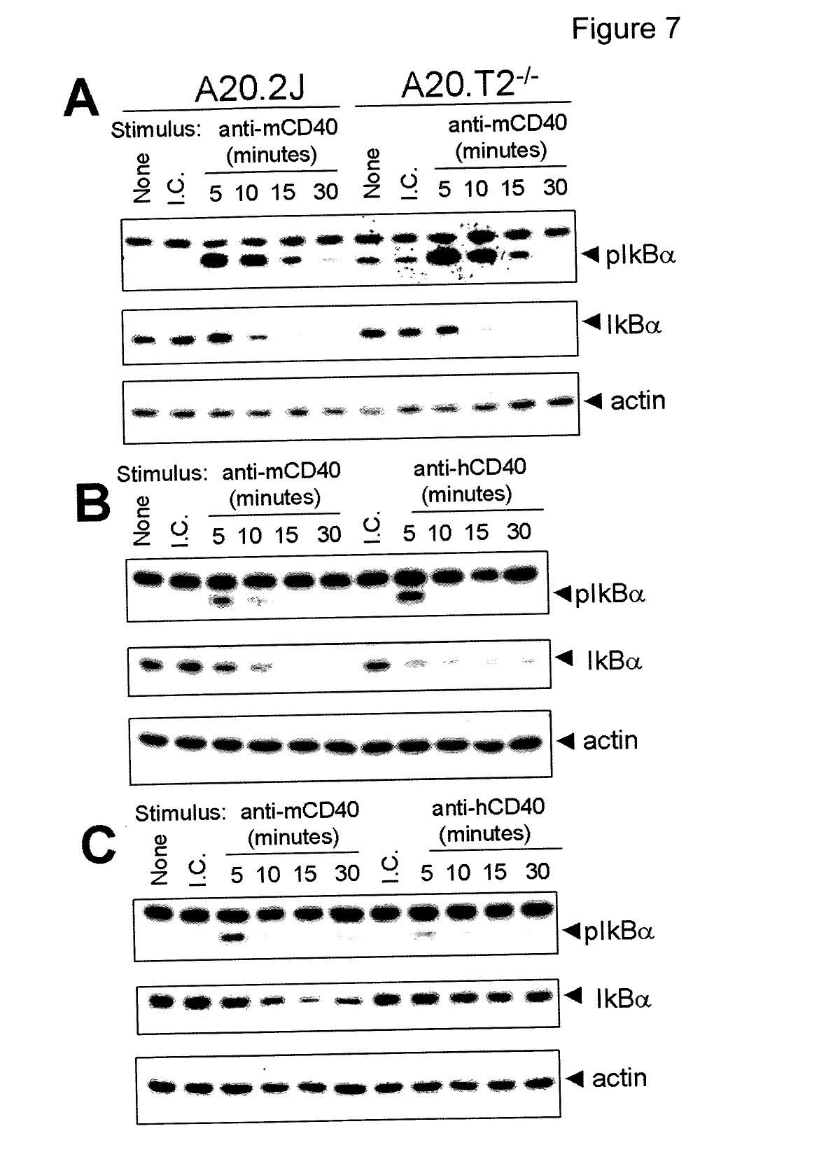

We found that the CD40-induced phosphorylation and degradation of IκBα (the first steps in the activation of NF-κB) in A20.2J cells was unaffected by the disruption of TRAF2 expression (FIG. 7A). Similar results were obtained with TRAF2-deficient CH12.LX cells (data not shown). These results and results from previous studies (Lomaga et al., 1999) suggest that TRAF6 may substitute for TRAF2 in activating NF-κB via CD40. To test this hypothesis, we examined NF-κB activation in A20.2J and A20.T2^{-/-} cells stably transfected with hmCD40 and hmCD40ΔT6 (clones with similar levels of hCD40 expression were used, data not shown). In cells expressing TRAF2, hmCD40ΔT6 induced robust phosphorylation and degradation of IκBα (FIG. 7B). However, stimulation of TRAF2^{-/-} cells through hmCD40ΔT6 resulted in weaker phosphorylation (upper panel) and little degradation (middle panel) of IκBα (FIG. 7C). These results indicate that neither TRAF2 nor an intact TRAF6 binding site are essential for the activation of NF-κB by CD40 in B cells. However, in the absence of both, TRAF1, 3, or 5 cannot act as substitutes.

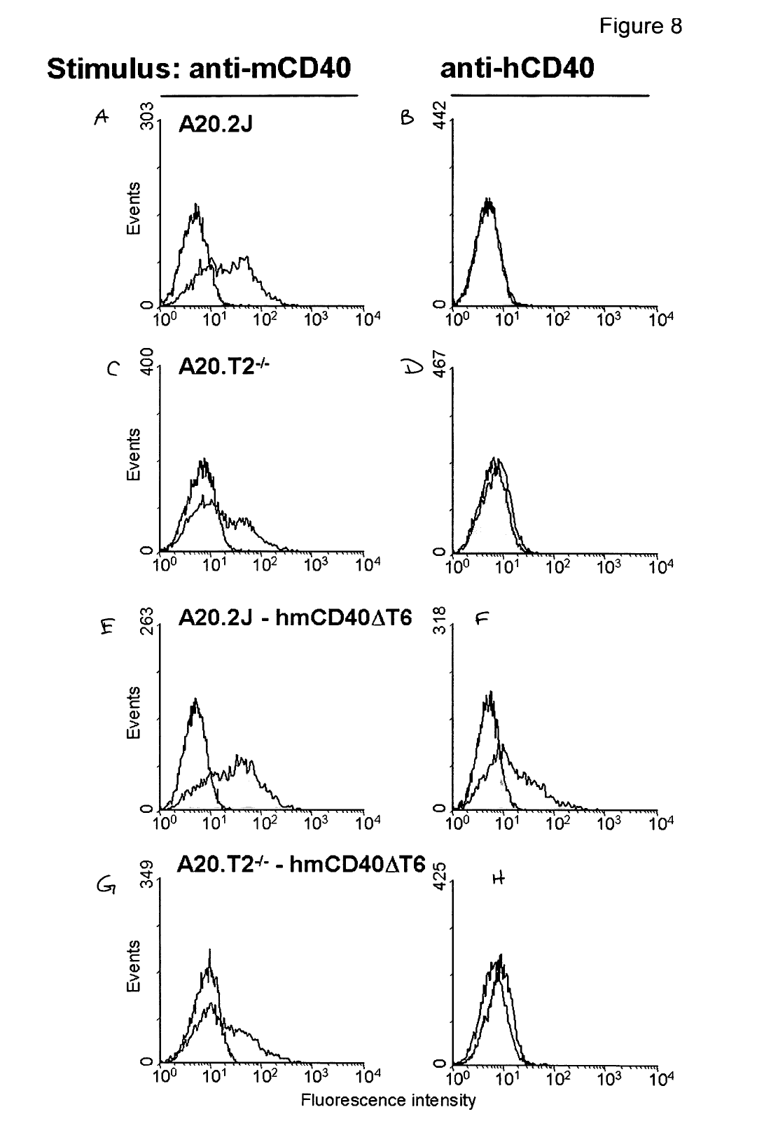

Engagement of CD40 on B lymphocytes has been shown to induce upregulation of a number of cell surface proteins, including CD80, that are critical to the activation of T cell-dependent humoral immune responses, but the roles of individual TRAFs in this process have again been unclear (Yasui et al., 2002; Hostager et al., 1996; Manning et al., 2002; Jalukar et al., 2000). We previously found that CD40-mediated CD80 upregulation in B cells is highly dependent upon NF-κB activation (Hsing et al. 1999). We thus considered the hypothesis that TRAFs 2 and 6 may also overlap in CD80 upregulation, via their redundancy in the NF-κB pathway, and that this could explain previous discrepancies in conclusions as to the role of either TRAF. To test this hypothesis, we stimulated TRAF2^{-/-} B cells through CD40 and examined expression of CD80. Although TRAF2-deficient cells appeared to have a partial defect in their ability to upregulate CD80 in response to CD40 signaling, the level of the defect falls within the range of variation observed among different clones of TRAF2-expressing A20.2J cells (FIG. 8). Although hmCD40ΔT6 stimulated upregulation of CD80 in A20.2J cells, it was unable to activate CD80 upregulation in A20.T2^{-/-} cells, suggesting redundant roles for TRAF2 and TRAF6 in this function, and supporting our hypothesis.

Discussion

Using TRAF2^{-/-} B cells, we were able to demonstrate novel roles of TRAF2 in CD40-mediated activation events. Analysis of TRAF function has been a complicated task because individual TNFR family members often interact with more than one TRAF family member, and the individual TRAFs often share binding sites. Due to these difficulties, the role of TRAF2 in CD40 signaling has been unclear, with various model systems leading to different conclusions (Nguyen et al., 1999; Jabara et al., 2002; Ahonen et al., 2002; Hsing et al., 1997). While transient high level expression of TRAFs in epithelial cells has been frequently used in characterizing TRAF function, it is clear that TRAF2 expressed under these conditions does not have the same CD40 binding activity or functional behavior as TRAF2 expressed at normal levels in B cells (Haxhinasto et al., 2002). The roles of the TRAFs have also been addressed with mutant CD40 transgenes expressed in CD40^{-/-} mice (Yasui et la., 2002, Ahonen et al., 2002). While this advance avoids TRAF overexpression and the viability problem associated with TRAF knockout animals, various aspects of this system complicate the conclusions drawn. First, the point mutation in CD40 intended to disrupt the binding of TRAF2 and TRAF3 is only partially effective (Haxhinasto et al., 2002). In addition, transgene expression varied considerably between mice expressing different CD40 constructs, leaving open the possibility that higher expression levels compensated for partial signaling defects in some of the CD40 mutants.

As demonstrated here, it is possible and practical to generate somatic cell lines deficient in individual TRAF molecules. This approach simplifies the analysis of TRAF function, and has led to new insights into the roles of TRAF2 in CD40 signaling. One unappreciated role of TRAF2 revealed by our experiments is its ability to promote the CD40-induced degradation of TRAF3. Our previous work, and work by a number of other investigators has demonstrated that signaling through CD40 and other members of the TNFR family results in the ubiquitination of TRAF molecules. The purpose of the ubiquitination is not entirely clear, although it may be important for the activation of certain signaling pathways. TRAF ubiquitination may also contribute to the regulation of signaling by targeting TRAFs for degradation (Brown et al., 2002; Brown et al., 2001). As we demonstrate, this targeting (likely mediated by ubiquitination) can occur in trans. An oncogenic viral mimic of CD40, LMP-1, illustrates this importance of this putative regulatory mechanism. The LMP-1 protein encoded by EBV binds strongly to TRAF3, which is presumably important for LMP-1 signaling. However, the interaction of TRAF2 with LMP-1 appears to be very weak, and we speculate that this arrangement may have evolved to limit the degradation of LMP-1-associated TRAF3. Further work is needed to better understand the role of TRAF3 (and the significance of its degradation) in signaling by CD40, LMP-1, and other receptors.

The contribution of TRAF2 to CD40-mediated NF-κB activation has been rather unclear, and the approach presented here has allowed clarification. Using TRAF2-deficient cells, we find that TRAF2 can contribute to NF-κB activation, but that other factors (potentially TRAF6) can largely substitute in its absence. Similar observations were made in regards to CD40-induced CD80 upregulation, which was previously shown to be highly NF-κB-dependent (Hsing et al., 1999). Considering the importance of CD40 signals to the activation of efficient humoral and cell-mediated immune responses, a certain amount of redundancy is understandable. The generation of cells deficient in both TRAF2 and TRAF6 will allow confirmation of the functional overlap between the two molecules.

TRAF2-deficient B cells have also allowed us to further our understanding of the multi-faceted role of TRAF2 in CD40-induced IgM secretion. In TRAF2^{-/-} cells, TNF-stimulated IgM secretion was virtually absent, confirming our previous hypothesis that CD40-induced TNF augments IgM secretion through TRAF2-dependent TNF receptor (CD120b) signaling (Hostager and Bishop, 2002). In contrast to the partially redundant roles of TRAF2 and TRAF6 in NF-κB activation and CD80 upregulation, our results show that the two TRAFs play unique and essential roles in IgM production. Although both TRAFs may participate in the NF-κB activation required for the induction of antibody secretion (Hsing et al., 1999), TRAF6 likely supplies an additional important signal as evidenced by the partial but significant defect in IgM secretion activated by hmCD40ΔT6 in cells expressing TRAF2. A particularly interesting and unexpected contribution of TRAF2 to CD40-mediated IgM secretion is its role in cooperative signaling between CD40 and the BCR. Previously, we found that a truncation (22 amino acids) of the CD40 CY domain disrupts TRAF2 binding, but has virtually no effect on the ability of CD40 to induce IgM secretion. Like wild-type CD40 signals, signaling by the mutant is augmented by BCR signals, resulting in enhanced IgM secretion. In TRAF2^{-/-} cells, cooperation of Wt CD40 with the BCR was defective. Cooperation was restored by Wt TRAF2, but also by a TRAF2 mutant that has been shown to be deficient in signaling activity. Together, these observations lead to the hypothesis that the binding of TRAF2 interferes with the binding of a negative regulatory factor to the CY domain of CD40 that would otherwise inhibit CD40-BCR synergy. Additional experiments with TRAF3-deficient CH12.LX cells indicate that TRAF3 or a TRAF3-associated factor is the inhibitor (P. Xie, S. Haxhinasto, G. Bishop, manuscript in preparation).

The targeted disruption of genes in somatic cell lines, while a potentially valuable tool in evaluating the roles of a variety of cellular proteins, has been used infrequently. The low frequency of homologous recombination in many somatic cell lines appears to be the major factor preventing greater exploitation of this approach. However, using a combination of technical strategies (see Experimental Procedures) we were able to surmount this obstacle. There are numerous signaling molecules whose depletion in the whole animal results in early lethality, or developmental defects so substantial that cells from these animals cannot be used to study normal cell function. Even “conditional knockout” animals often lose a particular protein in a given cell lineage from an early point in development. Our approach provides an alternative and complementary method that can be produced with much less time and expense, allows rapid transfection with desired molecules to test hypotheses and predictions, and targets genes specifically rather than using chemical mutagens that may produce additional unknown and undesired mutations.

Obviously, ours is but one approach that will ultimately lead to the elucidation of TNFR family signaling mechanisms. Alternatively, TRAF-specific RNA interference might be used to achieve similar goals (Hannon, 2002). However, it is important to note that residual protein expression must be expected using this technique. Considering the surprising amount of function retained by cells having even a small amount of TRAF2, the more complete disruption of gene expression achieved by homologous recombination is advantageous. Additionally, the development of improved animal models will be necessary as well to better understand the roles of the TRAFs in the complex interactions required for the generation of antigen-specific immune responses. Together, these approaches will allow us to better understand the complex interactions and functions of the TRAF molecules in signaling by TNFR family members.

### EXAMPLE 2

### Development and use of TRAF-Deficient Fibroblast Cell Lines

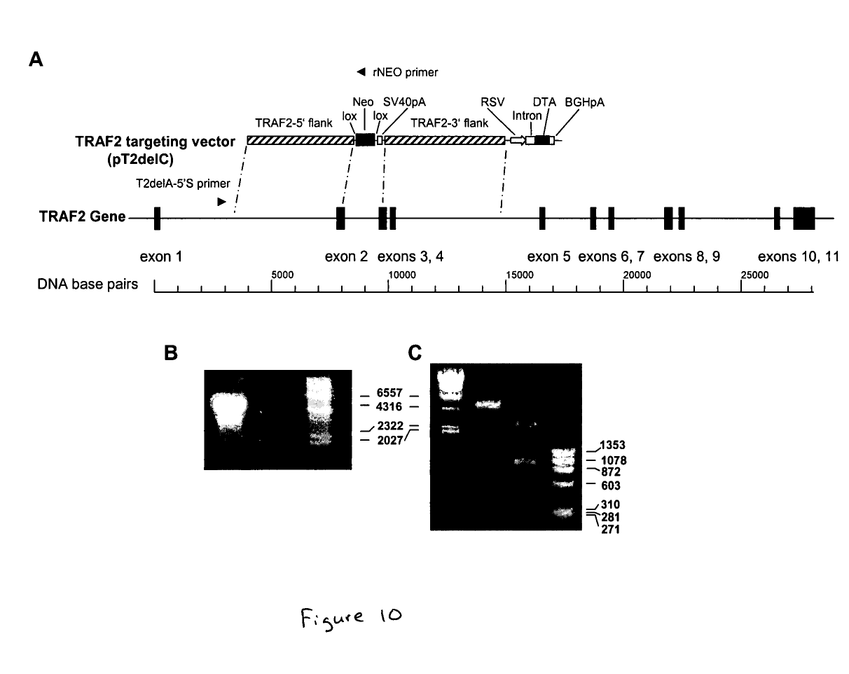

CH12.LX cells and NIH3T3 cells were transfected with a targeting vector designed to undergo homologous recombination with the gene coding for TRAF2, disrupting its expression. After transfection, cells were subcloned in medium containing G418 sulfate to select for cells in which the targeting vector had stably integrated. PCR screening of genomic DNA was used to identify clones in which the targeting vector had undergone homologous recombination (the PCR primers and results of the screening are shown in FIG. 10A). To confirm the PCR screening results from the NIH3T3 clone, the PCR product was digested with a restriction enzyme, resulting in the expected pattern of DNA fragments. The CH12.LX cells in which the targeting vector had undergone homologous recombination (FIG. 10A-10C) were subjected to a second round of targeting to disrupt the remaining copy of the gene, after which Western blotting confirmed that TRAF2 expression had been eliminated (not shown).

All publications, patents and patent applications referred to are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

Citations

Ahonen et al., Nat Immunol, 3, 451-6 (2002).

Arbones et al., Nat. Genet. 6, 90-97 (1994).

Baniyash and Eshhar, European Journal of Immunology, 14, 799-807 (1984).

Bennett et al., Nature, 393, 478-480 (1998).

Bishop and Haughton, Proc Natl Acad Sci, 83, 7410-4 (1986).

Bishop and Hostager (2001) Arch. Immunol. et Ther. Exper., 49, 129-137.

Bishop et al., Eur J Immunol, 25, 1230-8 (1995).

Bishop, J Immunol, 147, 1107-14 (1991).

Bourgeois et al. (2002) Science, 297, 2060-2063.

Brown et al., Science, 277, 831-834 (1997).

Brown et al., J Biol Chem 277 19433-8 (2002).

Brown et al. (2001) J. Exp. Med., 193, 943-954.

Busch and Bishop, J Immunol, 167, 5805-13 (2001).

Busch and Bishop, JI, 162, 2555-2561 (1999).

Cao et al. (1996) Nature, 383, 443-446.

Catlett et al. (2001) J. Immunol., 166, 6019-6024.

Deng et al. (2000) Cell, 103, 351-361.

Foy et al., Annual Review of Immunology, 14, 591-617 (1996).

Hamano et al., J Immunol, 129, 1403 (1982).

Hannon, G. J. (2002) Nature, 418, 244-251.

Hanson and Sedivy, Mol Cell Biol, 15, 45-51. (1995).

Hanson et al., Mol. Cell. Biol., 15, 45-51 (1995).

Haxhinasto et al I., J Immunol 169, 1145-9 (2002).

Heath et al., E. J. Immunol., 24, 1828-1834 (1994).

Hernan et al., Biotechniques, 28, 789-93 (2000).

Hostager and Bishop, J Immunol, 162 6307-6311 (1999).

Hostager and Bishop, J Immunol, 168, 3318-22 (2002).

Hostager et al., J. Biol. Chem, 275, 15392-15398 (2000).

Hostager et al., J. Immunol., 157, 1047-1053 (1996).

Hsing and Bishop, JI, 162, 2804-2811 (1999).

Hsing et al., Journal of Immunology, 159, 4898-906 (1997).

Hu et al., Journal of Biological Chemistry, 269, 30069-30072 (1994).

Inoue et al. (2000) Exp. Cell Res., 254, 14-24.

Ishida et al., Journal of Biological Chemistry, 271, 28745-8 (1996).

Ishida et al. (1996) Proc. Natl. Acad. Sci. (USA), 93, 9437-9442.

Jabara et al., Immunity, 17, 265 (2002).

Jalukar et al., J. Immunol., 164, 623-630 (2000).

Kim et al., J Immunol, 122, 549-54 (1979).

Lee et al., Immunity, 7, 703-713 (1997).

Li et al. (2002) Nature, 416, 345-347.

Liu et al., Cell, 87, 565-576 (1996).

Lomaga et al., Genes & Development, 13, 1015-1024 (1999).

Long et al., Hum Immunol, 31, 229-35 (1991).

Manning et al., Eur J Immunol, 32, 39-49. (2002).

Nakano et al. (1999) Proc. Nat. Acad. Sci. (USA), 96, 9803-9808.

Nakano et al. (1996) J. Biol. Chem., 271, 14661-14664.

Nguyen et al., Immunity, 11, 379-389 (1999).

Rothe et al., Science, 269, 1424-1427 (1995).

Sambrook and Russell, Molecular Cloning: A Laboratory Manual 3rd ed., Cold Spring Harbor Laboratory Press, Cold Spring Harbor, N.Y. (2001).

Sauer and Henderson, Nucleic Acids Research,147-61 (1989).

Sauer, Methods Enzymol, 225, 890-900 (1993).

Schoenberger et al., Nature 393, 480-483 (1998).

Sedivy and Dutriaux, Trends in Genetics, 15, 88-90 (1999).

Shi and Kehrl (2003) J. Biol. Chem., 278, 15429-15434.

Stout and Suttles, Immunology Today, 17, 487-492 (1996).

Takeuchi et al., Journal of Biological Chemistry, 271, 19935-42 (1996).

te Riele et al., Nature, 348, 649-51. (1990).

Turner et al., Mol. Biotech., 3:225 (1995).

Wagner et al. (2002) Proc. Nat. Acad. Sci. (USA), 99, 3782-3787.

Wang et al. (2001) Nature, 412, 346-351.

Yanagawa et al., Transgenic Research, 8, 215-221 (1999).

Yasui et al. (2002) Int. Immunol., 14, 319-329.

Yeh et al., Immunity, 7, 715-725 (1997).

What is claimed is:

1. A somatic cell gene targeting vector comprising:
(a) a gene targeting construct comprising a first cloning site operably linked to a DNA encoding a positive selection marker, a second cloning site and a first polyadenylation sequence, wherein the construct is promoterless; and
(b) an expression cassette comprising a promoter operably linked to DNA encoding a negative selection marker and a second polyadenylation sequence.

2. The vector of claim 1, wherein the gene targeting construct further comprises a first site-specific recombination sequence for a recombinase and a second site-specific recombination sequence for the recombinase, wherein the first and second site-specific recombination sequences flank the DNA encoding the positive selection marker.

3. The vector of claim 2, wherein the recombinase is Cre recombinase.

4. The vector of claim 2, wherein the first and second site-specific recombination sequences are loxP sequences.

5. The vector of claim 1, wherein the first cloning site comprises a first DNA segment that is homologous to a first genomic target sequence and the second cloning site comprises a second DNA segment that is homologous to a second genomic target sequence.

6. The vector of claim 1, wherein the positive selection marker is neomycin phosphotransferase.

7. The vector of claim 1, wherein the first polyadenylation sequence comprises a SV40 polyadenylation sequence.

8. The vector of claim 1, wherein the expression cassette comprises a weak promoter.

9. The vector of claim 1, wherein the expression cassette comprises a promoter that is a phosphoglycerate kinase (PGK) promoter or a modified Rous sarcoma virus (RSV) promoter.

10. The vector of claim 9, wherein the promoter is a modified RSV promoter.

11. The vector of claim 1, wherein the expression cassette comprises a BGH polyadenylation sequence.

12. The vector of claim 1, wherein the negative selection marker is HSV thymidine kinase or diphtheria toxin (DT-A).

13. A method for disrupting a gene of interest in a somatic cell, which method comprises introducing a targeting vector comprising a gene targeting construct comprising a first cloning site operably linked to a DNA encoding a positive selection marker, a second cloning site and a first polyadenylation sequence, wherein the construct is promoterless; and an expression cassette comprising a promoter operably linked to DNA encoding a negative selection marker and a second polyadenylation sequence, wherein the first cloning site comprises a first DNA segment that is homologous to a first genomic target sequence and the second cloning site comprises a second DNA segment that is homologous to a second genomic target sequence, into a somatic cell such that the first genomic target sequence and the second genomic target sequence recombine with the gene to yield a genetically altered cell.

14. The method of claim 13, wherein the vector recombines with the gene via homologous recombination

15. The method of claim 13, further comprising identifying the genetically altered cell, wherein the cell's genome comprises the construct and the positive selection marker is expressed.

16. The method of claim 13, wherein the somatic cell is a mammalian cell.

17. The method of claim 16, wherein the mammalian cell is a human cell.

18. The method of claim 13, further comprising introducing a double-stranded oligonucleotide into the somatic cell.

19. The method of claim 18, wherein the double-stranded oligonucleotide is 62 bp.

20. A method for disrupting a gene of interest in a somatic cell, which method comprises:
(a) introducing a targeting vector comprising a gene targeting construct comprising a first cloning site operably linked to a DNA encoding a positive selection marker, a second cloning site and a first polyadenylation sequence, wherein the construct is promoterless; and an expression cassette comprising a promoter operably linked to DNA encoding a negative selection marker and a second polyadenylation sequence, wherein the first cloning site comprises a first DNA segment that is homologous to a first genomic target sequence and the second cloning site comprises a second DNA segment that is homologous to a second genomic target sequence, into the somatic cell such that the first genomic target sequence and the second genomic target sequence recombine with the gene to yield a first genetically altered cell; and
(b) introducing a recombinase to the first genetically altered cell, such that the positive selection marker is removed from the construct to yield a second genetically altered cell.

21. The method of claim 20, wherein the vector recombines with the gene via homologous recombination.

22. The method of claim 20, further comprising identifying the first genetically altered cell, wherein the cell's genome comprises the construct and the positive selection marker is expressed.

23. The method of claim 22, further comprising identifying the second genetically altered cell.

24. The method of claim 20, wherein the somatic cell is a mammalian cell.

25. The method of claim 24, wherein the mammalian cell is a human cell.

26. The method of claim 20, further comprising introducing a double-stranded oligonucleotide into the somatic cell.

27. The method of claim 26, wherein the double-stranded oligonucleotide is 62 bp.

28. An isolated cell prepared by the method of claim 13.

29. An isolated cell prepared by the method of claim 20.

30. A somatic cell comprising the vector of claim 1.

31. The somatic cell of claim 30, wherein the cell is a B cell or a fibroblast cell.


SEQUENCE LISTING (11 sequences)

SEQ ID NO: 1 - DNA, 28 bp, Artificial Sequence
  A synthetic primer.
tagtcgaccgtgaagtggtgctgatggt

SEQ ID NO: 2 - DNA, 35 bp, Artificial Sequence
  A synthetic primer.
ctacccgggcagccatgtgagttacaacccccaca

SEQ ID NO: 3 - DNA, 32 bp, Artificial Sequence
  A synthetic primer.
atggtaccgtctatgaaaggggccagtgtagt

SEQ ID NO: 4 - DNA, 26 bp, Artificial Sequence
  A synthetic primer.
aatctagaacagggtgctctgcagtg

SEQ ID NO: 5 - DNA, 29 bp, Artificial Sequence
  A synthetic primer.
ttgtcgactgtgtgggggttgtaactcac

SEQ ID NO: 6 - DNA, 42 bp, Artificial Sequence
  A synthetic primer.
aatctagagtttaaactcagtcattcaatagacacaggcagc

SEQ ID NO: 7 - DNA, 30 bp, Artificial Sequence
  A synthetic primer.
tttggtacccagaactgtgctgcctgtgtc

SEQ ID NO: 8 - DNA, 35 bp, Artificial Sequence
  A synthetic primer.
aaggtaccaacagtttacagaaggtaagactaatg

SEQ ID NO: 9 - DNA, 22 bp, Artificial Sequence
  A synthetic primer.
cttagttttcacaatgccttcg

SEQ ID NO: 10 - DNA, 21 bp, Artificial Sequence
  A synthetic primer.
caatccatcttgttcagccat

SEQ ID NO: 11 - DNA, 25 bp, Artificial Sequence
  A synthetic primer.
gaattgaggtgtgatatggtctgtg